(19) United States
(12) Patent Application Publication
(10) Pub. No.: US 2023/0233601 A1
(43) Pub. Date: Jul. 27, 2023

(54) A HERBO-MINERAL METALLIC PHARMACEUTICAL KIT

(71) Applicant: Sadanand Prabhakar SARDESHMUKH, Pune (IN)

(72) Inventors: Sadanand Prabhakar SARDESHMUKH, Pune (IN); Vineeta Vasant DESHMUKH, Pune (IN)

(21) Appl. No.: 18/011,164

(22) PCT Filed: Jul. 5, 2021
(86) PCT No.: PCT/IB2021/056009

§ 371 Date: Dec. 17, 2022

(30) Foreign Application Priority Data
Jul. 3, 2020 (IN) 202021028308

(51) Int. Cl. A61K 33/242; A61K 33/10; A61K 31/718; A61K 36/71; A61K 47/36; A61K 35/618; A61K 35/614; A61K 35/35; A61K 9/20; A61P 35/00

(52) U.S. Cl. CPC A61K 33/242; A61K 33/10; A61K 31/718; A61K 36/71; A61K 47/36; A61K 35/618; A61K 35/614; A61K 35/35; A61K 9/2072; A61K 9/2095; A61P 35/00

(57) ABSTRACT

The present disclosure relates to a herbo-mineral metallic pharmaceutical kit and a process for its preparation. The herbo-mineral metallic pharmaceutical kit comprises Suvarna Bhasmadi Vati (SBV), Mouktikyukta Kamdudha Vati (MKV), Trailokyachintamani Rasa Vati (TCR), Kumari Kalpa Vati (KKV) and Khadirarishta (KHA). The herbo-mineral metallic pharmaceutical kit of the present disclosure can be used for delaying and/or controlling the recurrence or occurrence of breast and/or ovarian cancer in second breast and/or ovary or metastasis. The herbo-mineral metallic pharmaceutical compositions of the kit assist in reducing oxidative stress, improving immune status and quality of life and maintaining a disease-free survival in patients who are genetically predisposed to breast or ovarian cancer.

This application is a National Phase of PCT Patent Application No PCT/162021/056009 having International filing date of Jul. 5, 2021, which claims the benefit of priority of Indian Patent Application No. 202021028308, filed Jul. 3, 2020, the contents of which are all incorporated herein by reference in their entirety.

### FIELD

The present disclosure relates to a herbo-mineral metallic pharmaceutical kit. Particularly, the present disclosure relates to a herbo-mineral metallic pharmaceutical kit for delaying and/or controlling the recurrence or occurrence of breast and/or ovarian cancer in genetically predisposed individuals.

### Abbreviations

WBC: White blood cells.

SGOT: Serum glutamic oxaloacetic transaminase.

SGPT: Serum glutamic pyruvic transaminase.

CRP: C-Reactive Protein

RSR: Respective survival rate

QoL: Quality of Life

QLQ C 30: Quality of Life Questionnaire

BRCA1 or BRCA2: BRCA is a human tumor suppressor gene (also known as a caretaker gene).

TP53: Tumor protein p53 (or p53).

### Definitions

As used in the present disclosure, the following terms are generally intended to have the meaning as set forth below, except to the extent that the context in which they are used indicate otherwise.

Suvarna bhasma: The term “Suvarna bhasma” refers to “incinerated gold” prepared by incinerating gold in accordance with the present disclosure. The term “Suvarna bhasma” referred in the present disclosure is not the same, as used in the Ayurveda. Suvarna refers to 24 carat gold.

Mouktik bhasma: The term “Mouktik bhasma” refers to “incinerated pearl”, natural or cultured, prepared by incinerating pearl in accordance with the present disclosure. The term “Mouktik bhasma” referred in the present disclosure is not the same, as used in the Ayurveda.

Roupya bhasma: The term “Roupya bhasma” refers to “incinerated silver”. The term

“Roupya bhasma” referred in the present disclosure is not the same, as used in the Ayurveda.

Heerak bhasma: The term “Heerak bhasma” refers to “incinerated diamonds”. The term “Heerak bhasma” referred in the present disclosure is not the same, as used in the Ayurveda.

Tamra bhasma: The term “Tamra bhasma” refers to “incinerated copper”. The term “Tamra bhasma” referred in the present disclosure is not the same, as used in the Ayurveda.

Abhrak bhasma: The term “Abhrak bhasma” refers to “incinerated mica”. The term “Abhrak bhasma” referred in the present disclosure is not the same, as used in the Ayurveda.

Loha bhasma: The term “Loha bhasma” refers to “incinerated iron”. The term “Loha bhasma” referred in the present disclosure is not the same, as used in the Ayurveda.

Vaikrant bhasma: The term “Vaikrant bhasma” refers to “incinerated tourmaline”. The term “Vaikrant bhasma” referred in the present disclosure is not the same, as used in the Ayurveda.

Rasasindoor: The term “Rasasindoor” refers to “red sulphide of mercury”. The term “Rasasindoor” referred in the present disclosure is not the same, as used in the Ayurveda.

Shuddha Hartaal: The term “Shuddha Hartaal” refers to “processed orpiment”. The term “Shuddha Hartaal” referred in the present disclosure is not the same, as used in the Ayurveda.

Shuddha Manahshila: The term “Shuddha Manahshila” refers to “processed realgar”. The term “Shuddha Manahshila” referred in the present disclosure is not the same, as used in the Ayurveda.

Kajjali: The term “Kajjali” refers to “black sulphide of mercury”. The term “Kajjali” referred in the present disclosure is not the same, as used in the Ayurveda

Shankha bhasma: The term “Shankha bhasma” refers to “incinerated Conch shell”. The term “Shankha bhasma” referred in the present disclosure is not the same, as used in the Ayurveda.

Shouktik bhasma: The term “Shouktik bhasma” refers to “incinerated empty pearl shell”. The term “Shouktik bhasma” referred in the present disclosure is not the same, as used in the Ayurveda.

Kapardikbhasma: The term “Kapardik bhasma” refers to “incinerated Cowries”. The term “Kapardikbhasma” referred in the present disclosure is not the same, as used in the Ayurveda.

Pravala bhasma: The term “Pravala bhasma” refers to “incinerated coral”. The term “Pravala bhasma” referred in the present disclosure is not the same, as used in the Ayurveda.

Shuddha Tankan: The term “Shuddha Tankan” refers to “dehydrated sodium borate”. The term “Shuddha Tankan” referred in the present disclosure is not the same, as used in the Ayurveda.

Shuddha Vatsanabh: The term “Shuddha Vatsanabh” refers to “purified Aconitum ferox” or related species. The term “Shuddha Vatsanabh” referred in the present disclosure is not the same, as used in the Ayurveda.

Shuddha Gairik: The term “Gairik” is a natural clay earth pigment which is a mixture of ferric oxide and varying amounts of clay and sand. “Shuddha Gairik” refers to “processed Gairik”, prepared by roasting Gairik in ghee obtained from cow's milk. The term “Shuddha Gairik” referred in the present disclosure is not the same, as used in the Ayurveda.

Guduchi Sattva: The term “Guduchi Sattva” refers to an extract comprising mainly starch of Tinospora cordifolia/sinensis/crispa/glabra prepared by alcoholic, hydro-alcoholic or aqueous extraction method. The term “Guduchi Sattva” referred in the present disclosure is not the same, as used in the Ayurveda

Gud: The term “Gud” refers to “Jaggery” prepared from “Saccharrum officinarum”.

Sunthi: The term “Sunthi” refers to dry ginger “Zingiber officinale”.

Marich: The term “Marich” refers to “Piper nigrum”.

Pippali: The term “Pippali” refers to “Piper longum”.

Bhumiamalaki: The term “Bhumiamalaki” refers to “Phyllanthus nirurf” or related species.

Bibhitaki: The term “Bibhitaki” refers to “Terminalia bellerica”.

Haritaki: The term “Haritaki” refers to “Terminalia chebula”.

Kutaki: The term “Kutaki” refers to “Picrorrhiza kurroa”.

Vidang: The term “Vidang” refers to “Embelia ribes” or related species.

Musta: The term “Musta” refers to “Cyperus rotundus” or related species.

Sharpunkha: The term “Sharpunkha” refers to “Tephrosia purpurea” or pauciflora.

Kumari: The term “Kumari” refers to “Aloe vera or barbadensis” or related species.

Chitrak: The term “Chitrak” refers to “Plumbago zeylanica” or related species.

Nirgundi: The term “Nirgundi” refers to “Vitex negundo” or related species.

Surana: The term “Surana” refers to “Amorphophyllus campanulatus”.

Arka dugdha: The term “Arka dugdha” refers to latex of “Calotropis procera” or related species.

Snuhi dugdha: The term “Snuhi dugdha” refers to latex of “Euphorbia nerifolia” or related species.

Shigru: The term “Shigru” refers to “Moringa oliefera”.

Ardrak: The term “Ardrak” refers to fresh ginger “Zingiber officinale”.

Nimbuk: The term “Nimbuk” refers to “Citruslimon” or related species.

Jatiphal: The term “Jatiphal” refers to “Myristica fragrans”.

Lavang: The term “Lavang” refers to “Syzygium aromaticum”.

Amalaki: The term “Amalaki” refers to “Emblica officinalis”.

Tamalpatra: The term “Tamalpatra” refers to “Cinnamomum tamala”.

Bruhad ela: The term “Bruhad ela” refers to “Amommum subulatum”.

Dhataki: The term “Dhataki” refers to “Woodfordia fruticosa”.

Dalchini: The term “Dalchini” refers to “Cinnamomum zeylanicum”.

Kankol: The term “Kankol” refers to “Piper cubeba”.

Nagkeshar: The term “Nagkeshar” refers to “Mesua ferrea” or “Calophyllum inophyllum”.

Khadir: The term “Khadir” refers to “Acacia catechu” or related species.

Daruharidra: The term “Daruharidra” refers to “Berberis aristata” or related species.

Devdaru: The term “Devdaru” refers to “Cedrus deodara”.

Vati: The term “Vati” refers to a method of medicine preparation in which herbs, minerals, and metallic compounds are compressed into tablet form.

Trituration: The term “trituration” refers to either reducing the particle size of a substance or production of a homogeneous material by mixing component materials thoroughly or wet grinding any material with a liquid media like fresh juice or decoction etc.

Incineration: The term “incineration” refers to heating solid dried materials by using cow-dung cakes or muffle furnace.

Fermentation: Fermentation is a process to allow specific bacteria to grow in a specific medium at specific temperature and time.

Candle Test: Candle test confirms end point of fermentation in which glowing of the flame takes place due to absence of carbon dioxide and presence of oxygen in the fermentation vessel.

Karnofsky score: The term “Karnofsky score” refers to the Karnofsky Performance Scale Index allows patients to be classified as to their functional impairment

Symptom score: Symptom score of QLQ is indicative of symptomatology; hence decrease in symptom score represents both decrease in disease related symptoms and adverse effects of conventional treatment.

Function score: Functional score of QLQ signifies status of routine physical activities. Increase in functional scores represents improvement in QoL.

Global score: Global score of QLQ represents overall well-being of a patient. Increase in global scores represents improvement in QoL.

### BACKGROUND

The background information herein below relates to the present disclosure but is not necessarily a prior art.

A large number of hereditary cancer syndromes are identified and as per the American College of Medical Genetics (ACMG) recommendations, there are at least 25 cancer predisposing genes which can be tested for the presence of germline mutations. The common hereditary syndromes include Hereditary Breast and Ovarian Cancer (HBOC) syndrome due to mutation in BRCA1/2 genes, Lynch syndrome due to mutation in Mismatch Repair (MMR) genes and Li-Fraumeni Syndrome (LFS) due to TP53 gene mutation. It is known that development of breast or ovarian cancer is due to germ-line mutations and can be seen in 14.1% of patients suffering from breast or ovarian cancer. A majority of patients suffering from Hereditary Breast/Ovarian Cancer (HBOC) syndrome have germline mutations in BRCA1 or BRCA2 genes. The overall prevalence of BRCA1/2 mutations is estimated to be from 1 in 400 to 1 in 800 in human population. Healthy women carrying BRCA1/2 mutations have a high life time risk of developing breast and or ovarian cancer. Germline mutations in these genes confer a very high (70-80%) life-time risk for developing breast and or ovarian cancer. The lifetime risk of developing breast cancer among female mutation carriers is 82% for BRCA1 mutation carriers while the lifetime risk of developing ovarian cancer is 54% for BRCA1 and 23% for BRCA2 mutation carriers. BRCA1 is estimated to confer a breast cancer risk of 54% by age 60 years and an ovarian cancer risk of 30% by age 60 years.

Breast cancer patients carrying BRCA1/2 mutation have limited life expectancy and limited effectiveness of therapy. They also have a higher risk of developing malignancy in ovary and vice a versa. Breast cancer patients carrying BRCA1/2 mutation also have additional risk of developing contra lateral malignancy in second breast. Therefore, these patients are advised prophylactic surgery to remove ovaries and sometimes uninvolved breast. However salphingo oophorectomy (removal of ovaries) results in premature menopause and its associated effect on quality of life, bone health and several other parameters. While contralateral prophylactic mastectomy (removal of uninvolved breast) can reduce the breast cancer risk to 75% to 95%. Hormonal therapy using Tamoxifen or other hormonal treatment offers partial protection from metastasis/recurrence but it has its own long-term toxicity including the risk of endometrial cancer.

This underscores the need for developing and evaluating new strategies with superior efficacy or compliance in preventing occurrence of cancer and preventing or delaying the onset of second malignancy and increasing cancer free survival with good physical and mental quality of life.

Therefore, there is felt a need to provide a herbo-mineral metallic combination in the form of a herbo-mineral metallic pharmaceutical kit that mitigates the afforestated problems.

### OBJECTS

Some of the objects of the present disclosure, which at least one embodiment herein satisfies, are as follows:

An object of the present disclosure is to ameliorate one or more problems of the prior art or to at least provide a useful alternative.

Another object of the present disclosure is to provide a herbo-mineral metallic pharmaceutical kit.

Yet another object of the present disclosure is to provide a herbo-mineral metallic pharmaceutical kit for delaying and/or controlling the recurrence or occurrence of breast and/or ovarian cancer in second breast and/or ovary or metastasis in genetically predisposed patients.

Still another object of the present disclosure is to provide a herbo-mineral metallic pharmaceutical kit for maintaining biochemical and clinical parameters of genetically predisposed patients of breast and/or ovarian cancers.

Another object of the present disclosure is to provide a herbo-mineral metallic pharmaceutical kit for reducing oxidative stress in genetically predisposed patients of breast and/or ovarian cancer.

Yet another object of the present disclosure is to provide a herbo-mineral metallic pharmaceutical kit for improving the immune response in genetically predisposed patients of breast and/or ovarian cancer.

Still another object of the present disclosure is to provide a herbo-mineral metallic pharmaceutical kit for improving the quality of life in genetically predisposed patients of breast and/or ovarian cancer.

Other objects and advantages of the present disclosure will be more apparent from the following description, which is not intended to limit the scope of the present disclosure.

### SUMMARY

The present disclosure provides a herbo-mineral metallic pharmaceutical kit for delaying and/or controlling the recurrence or occurrence of breast and/or ovarian cancer.

In one aspect, the present disclosure provides a herbo-mineral metallic pharmaceutical kit comprising a first container containing Suvarna Bhasmadi Vati (SBV) in a solid dosage form in an amount in the range of 300 mg/day to 500 mg/day, a second container containing Mouktikyukta Kamdudha Vati (MKV) in solid dosage form in an amount in the range of 800 mg/day to 1200 mg/day, a third container containing Trailokya chintamani Rasa Vati (TCR) in solid dosage form in an amount in the range of 200 mg/day to 300 mg/day, a fourth container containing Kumari Kalpa Vati (KKV) in solid dosage form in an amount in the range of 1600 mg/day to 2400 mg/day and fifth container containing Khadirarishta (KHA) in liquid dosage form in an amount in the range of 10 ml/day to 30 ml/day.

The first container contains Suvarna Bhasmadi Vati (SBV) in solid dosage form, which is prepared from Suvarna bhasma in an amount ranging from 2 wt % to 7 wt % of the total weight of SBV, Mouktik bhasma in an amount ranging from 20 wt % to 35 wt % of the total weight of SBV, Guduchisattva in an amount ranging from 45 wt % to 60 wt % of the total weight of SBV and at least one first excipient in an amount ranging from 5 wt % to 30 wt % of the total weight of SBV.

The second container contains Mouktikyukta Kamdudha Vati (MKV) in solid dosage form, which is prepared from Mouktik bhasma in an amount ranging from 10 wt % to 14 wt % of the total weight of MKV, Shankha bhasma in an amount ranging from 10 wt % to 14 wt % of the total weight of MKV, Shouktik bhasma in an amount ranging from 10 wt % to 14 wt % of the total weight of MKV, Kapardik bhasma in an amount ranging from 10 wt % to 14 wt % of the total weight of MKV, Praval bhasma in an amount ranging from 10 wt % to 14 wt % of the total weight of MKV, Guduchi sattva in an amount ranging from 10 wt % to 14 wt % of the total weight of MKV, Shuddha Gairik in an amount ranging from 10 wt % to 14 wt % of the total weight of MKV, and at least one excipient in an amount ranging from 10 wt % to 30 wt % of the total weight of MKV.

The third container contains Trailokya chintamani Rasa Vati (TCR) in solid dosage form, which is prepared from Kajjali in an amount ranging from 0.012 wt % to 0.016 wt % of the total weight of TCR, Suvarna bhasma in an amount ranging from 0.006 wt % to 0.008 wt % of the total weight of TCR, Roupya bhasma in an amount ranging from 0.006 wt % to 0.008 wt % of the total weight of TCR, Heerak bhasma in an amount ranging from 0.006 wt % to 0.008 wt % of the total weight of TCR, Loha bhasma in an amount ranging from 0.006 wt % to 0.008 wt % of the total weight of TCR, Abhrak bhasma in an amount ranging from 0.006 wt % to 0.008 wt % of the total weight of TCR, Tamra bhasma in an amount ranging from 0.006 wt % to 0.008 wt % of the total weight of TCR, Shankha bhasma in an amount ranging from 0.006 wt % to 0.008 wt % of the total weight of TCR, Mouktik bhasma in an amount ranging from 0.006 wt % to 0.008 wt % of the total weight of TCR, Praval bhasma in an amount ranging from 0.006 wt % to 0.008 wt % of the total weight of TCR, Shuddha Hartaal in an amount ranging from 0.006 wt % to 0.008 wt % of the total weight of TCR, Shuddha Manahshila in an amount ranging from 0.006 wt % to 0.008 wt % of the total weight of TCR, Shuddha Kapardik in an amount ranging from 2.7 wt % to 4.7 wt % of the total weight of TCR, Rasasindoor in an amount ranging from 3 wt % to 5 wt % of the total weight of TCR, Vaikrant bhasma in an amount ranging from 0.75 wt % to 1.25 wt % of the total weight of TCR, Shuddha Tankan in an amount ranging from 5.1 wt % to 7.1 wt % of the total weight of TCR, Shuddha Vatsanabh in an amount ranging from 5.1 wt % to 7.1 wt % of the total weight of TCR, Marich powder in an amount ranging from 5.1 wt % to 7.1 wt % of the total weight of TCR, Suntha powder in an amount ranging from 1.4 wt % to 1.6 wt % of the total weight of TCR, Pippali powder in an amount ranging from 1.4 wt % to 1.6 wt % of the total weight of TCR, Lavang powder in an amount ranging from 1.4 wt % to 1.6 wt % of the total weight of TCR, Jatiphal powder in an amount ranging from 1.4 wt % to 1.6 wt % of the total weight of TCR, Haritaki powder in an amount ranging from 1.4 wt % to 1.6 wt % of the total weight of TCR, all the extracts from Chitrak, Shigru, Ardrak and Nimbuk in an amount ranging from 54.5 wt % to 56 wt % of the total weight of TCR, and at least one excipient in an amount ranging from 5 wt % to 15 wt % of the total weight of the TCR. During preparation, intermittent triturations are performed with Arka dugdha in an amount ranging from 0.13 to 0.17% v/v, Nirgundi fresh juice in an amount ranging from 0.13 to 0.17% v/v, Surana fresh juice in an amount ranging from 0.13 to 0.17% v/v, Snuhi Kshir in an amount ranging from 0.13 to 0.17% v/v, Chitrak decoction in an amount ranging from 10.5 to 14.5% v/v, Shigru fresh juice in an amount ranging from 3.2 to 3.6% v/v, Ardrak fresh juice in an amount ranging from 30.75 to 34.75% v/v and Nimbuk fresh juice in an amount ranging from 48.75 to 52.75% v/v of the total liquid media used for trituration.

The fourth container contains Kumari Kalpa Vati (KKV) in solid dosage form, which is prepared from Suntha powder in an amount ranging from 7 wt % to 9 wt % of the total weight of KKV, Marich powder in an amount ranging from 7 wt % to 9 wt % of the total weight of KKV, Pippali powder in an amount ranging from 7 wt % to 9 wt % of the total weight of KKV, Bhumiamalaki powder in an amount ranging from 7 wt % to 9 wt % of the total weight of KKV,

Bibhitaki powder in an amount ranging from 7 wt % to 9 wt % of the total weight of KKV, Haritaki powder in an amount ranging from 7 wt % to 9 wt % of the total weight of KKV, Musta powder in an amount ranging from 7 wt % to 9 wt % of the total weight of KKV, Vidang powder in an amount ranging from 7 wt % to 9 wt % of the total weight of KKV, Sharpunkha powder in an amount ranging from 7 wt % to 9 wt % of the total weight of KKV, Kutki powder in an amount ranging from 7 wt % to 9 wt % of the total weight of KKV, dried Kumaripulp in an amount ranging from 1.75 wt % to 2.25 wt % of the total weight of KKV, and at least one excipient in an amount ranging from 8 wt % to 28 wt % of the total weight of KKV.

The fifth container contains Khadirarishta (KHA) in liquid dosage form, which is prepared from Khadir coarse powder in an amount ranging from 8.75 wt % to 12.75 wt % of the total weight of KHA, Devdaru coarse powder in an amount ranging from 8.75 wt % to 12.75 wt % of the total weight of KHA, Daruharidra coarse powder in an amount ranging from 2.3 wt % to 6.3 wt % of the total weight of KHA, Haritaki coarse powder in an amount ranging from 1 wt % to 1.5 wt % of the total weight of KHA, Bibhitaki coarse powder in an amount ranging from 1 wt % to 1.5 wt % of the total weight of KHA, Amalaki coarse powder in an amount ranging from 1 wt % to 1.5 wt % of the total weight of KHA, Jatiphal powder in an amount ranging from 0.19 wt % to 0.23 wt % of the total weight of KHA, Tamalpatra powder in an amount ranging from 0.19 wt % to 0.23 wt % of the total weight of KHA, Bruhad Ela powder in an amount ranging from 0.19 wt % to 0.23 wt % of the total weight of KHA, Lavang powder in an amount ranging from 0.19 wt % to 0.23 wt % of the total weight of KHA, Dalchini powder in an amount ranging from 0.19 wt % to 0.23 wt % of the total weight of KHA, Kankol powder in an amount ranging from 0.19 wt % to 0.23 wt % of the total weight of KHA, Nagkeshar powder in an amount ranging from 0.19 wt % to 0.23 wt % of the total weight of KHA, Pippali powder in an amount ranging from 0.19 wt % to 0.23 wt % of the total weight of KHA, jaggery crushed in an amount ranging from 60 wt % to 70 wt % of the total weight of KHA and Dhataki flowers in an amount ranging from 3.1 wt % to 3.5 wt % of the total weight of KHA.

In another aspect, the present disclosure provides a process of preparing the herbo-mineral metallic pharmaceutical kit. The herbo-mineral metallic pharmaceutical kit comprises Suvarna Bhasmadi Vati (SBV) in solid dosage form, Mouktikyukta Kamdudha Vati (MKV) in solid dosage form, Trailokya chintamani Rasa Vati (TCR) in solid dosage form, Kumari Kalpa Vati (KKV) in solid dosage form and Khadiraishta (KHA) in liquid dosage form.

### BRIEF DESCRIPTION OF THE ACCOMPANYING DRAWING

The present disclosure will now be described with the help of the accompanying drawing, in which:

FIG. 1a: Illustrates a graphical representation of effect on hemoglobin in BRCA positive breast cancer patients treated with the herbo-mineral metallic pharmaceutical composition;

FIG. 1b: Illustrates a graphical representation of effect on WBC count in BRCA positive breast cancer patients treated with the herbo-mineral metallic pharmaceutical composition;

FIG. 1c: Illustrates a graphical representation of effect on platelet count in BRCA positive breast cancer patients treated with the herbo-mineral metallic pharmaceutical composition;

FIG. 2a: Illustrates a graphical representation of effect on serum bilirubin in BRCA positive breast cancer patients treated with the herbo-mineral metallic pharmaceutical composition;

FIG. 2b: Illustrates a graphical representation of effect on SGOT in BRCA positive breast cancer patients treated with the herbo-mineral metallic pharmaceutical composition;

FIG. 2c: Illustrates a graphical representation of effect on SGPT in BRCA positive breast cancer patients treated with the herbo-mineral metallic pharmaceutical composition;

FIG. 2d: Illustrates a graphical representation of effect on serum alkaline phosphatase in BRCA positive breast cancer patients treated with the herbo-mineral metallic pharmaceutical composition;

FIG. 3a: Illustrates a graphical representation of effect on serum creatinine in BRCA positive breast cancer patients treated with the herbo-mineral metallic pharmaceutical composition;

FIG. 3b: Illustrates a graphical representation of effect on serum urea in BRCA positive breast cancer patients treated with the herbo-mineral metallic pharmaceutical composition;

FIG. 4: Illustrates a graphical representation showing the effect on CRP level in BRCA positive breast cancer patients treated with the herbo-mineral metallic pharmaceutical composition;

FIG. 5: Illustrates a graphical representation showing the effect on CA125 level in BRCA positive breast cancer patients treated with the herbo-mineral metallic pharmaceutical composition;

FIG. 6a: Illustrates a graphical representation of effect on IL-1β in BRCA positive breast cancer patients treated with the herbo-mineral metallic pharmaceutical composition;

FIG. 6b: Illustrates a graphical representation of effect on IL-6 in BRCA positive breast cancer patients treated with the herbo-mineral metallic pharmaceutical composition;

FIG. 6c: Illustrates a graphical representation of effect on IL-8 in BRCA positive breast cancer patients treated with the herbo-mineral metallic pharmaceutical composition;

FIG. 6d: Illustrates a graphical representation of effect on IL-10 in BRCA positive breast cancer patients treated with the herbo-mineral metallic pharmaceutical composition;

FIG. 7a: Illustrates a graphical representation showing the effect on superoxide dismutase (SOD) in BRCA positive breast cancer patients treated with the herbo-mineral metallic pharmaceutical composition;

FIG. 7b: Illustrates a graphical representation showing the effect on catalase in BRCA positive breast cancer patients treated with the herbo-mineral metallic pharmaceutical composition;

FIG. 7c: Illustrates a graphical representation showing the effect on glutathione in BRCA positive breast cancer patients treated with the herbo-mineral metallic pharmaceutical composition;

FIG. 8: Illustrates a graphical representation showing the effect on Karnofsky score and weight in BRCA positive breast cancer patients treated with the herbo-mineral metallic pharmaceutical composition;

FIG. 9: Illustrates a graphical representation showing the effect on Quality of Life (QLQ) in BRCA positive breast cancer patients treated with the herbo-mineral metallic pharmaceutical composition.

### DETAILED DESCRIPTION

Embodiments, of the present disclosure, will now be described with reference to the accompanying drawings.

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

Females with germline mutation in BRCA1/2 or TP53 mutation have 60-80% lifetime risk of breast cancer and 20-40% risk of ovarian cancer. Moreover, these females are at 40-50% risk of developing bilateral or contralateral breast cancer within 15 years of diagnosis of first breast cancer. Males with BRCA1/2 mutation have 15-25% risk of developing either prostate cancer or male breast cancer. Due to the low acceptability of preventive surgery and unestablished role of Tamoxifen as a chemo-preventive agent for breast cancer, there is an urgent need to explore other non-surgical methods for preventing new cancers in these genetically predisposed individuals. Herbo-mineral metallic medicines are known to improve general well-being or Quality of Life (QoL) of the patients and hence can be used in the form of a pharmaceutical composition.

Manifestation of breast or ovarian cancer in healthy carriers of BRCA 1/2 mutation or development of contralateral breast cancer or ovarian cancer in patients carrying BRCA1/2 mutation are the consequences of hampered immune system and increased oxidative stress due to various external factors like unhealthy diet, abnormal lifestyle, addiction and exposure to carcinogens. These two biochemical changes are effectively corrected by a selected combination of pharmaceutical compositions of the present disclosure, in the form of a herbo-mineral metallic pharmaceutical kit.

The present disclosure provides a herbo-mineral metallic pharmaceutical kit for reducing oxidative stress, improving immune status and quality of life (QoL), and maintaining a disease-free survival for patients who are genetically predisposed to breast or ovarian cancer, including prevention of bilateral or contralateral breast cancer or ovarian cancer or metastasis in genetically predisposed cancer patients having BRCA 1, BRCA 2 and TP53 mutation.

The present disclosure also provides a process for preparing the herbo-mineral metallic pharmaceutical kit.

The herbo-mineral metallic pharmaceutical kit of the present disclosure comprises a first container containing Suvarna Bhasmadi Vati (SBV) in solid dosage form in an amount in the range of 300 mg/day to 500 mg/day, a second container containing Mouktikyukta Kamdudha Vati (MKV) in solid dosage form in an amount in the range of 800 mg/day to 1200 mg/day, a third container containing Trailokya chintamani Rasa Vati (TCR) in solid dosage form in an amount in the range of 200 mg/day to 300 mg/day, a fourth container containing Kumari Kalpa Vati (KKV) in solid dosage form in an amount in the range of 1600 mg/day to 2400 mg/day and fifth container containing Khadirarishta (KHA) in liquid dosage form in an amount in the range of 10 ml/day to 30 ml/day.

The first container contains Suvarna Bhasmadi Vati (SBV) in solid dosage form, which is prepared from Suvarna bhasma in an amount ranging from 2 wt % to 7 wt % of the total weight of SBV, Mouktik bhasma in an amount ranging from 20 wt % to 35 wt % of the total weight of SBV, Guduchi sattva in an amount ranging from 45 wt % to 60 wt % of the total weight of SBV, and at least one first excipient in an amount ranging from 5 wt % to 30 wt % of the total weight of SBV.

Guduchi is the common name for Tinospora plant belonging to family Menispermaceae. The Tinospora (Guduchi) plant is selected from Tinospora cordifolia and Tinospora sinensis. Particularly the stem of the plant is used for making the Sattva. Tinospora cordifolia is indigenous to the tropical areas of India, Myanmar, and Sri Lanka while Tinospora sinensis is found in India, China, Sri Lanka, Nepal, Cambodia, Thailand, Vietnam, and Myanmar. In the present disclosure, Tinospora cordifolia and Tinospora sinensis is obtained from Bharatiya Sanskriti Darshan Trust (BSDT), Wagholi, Pune.

In an embodiment of the present disclosure, Suvarna Bhasmadi Vati (SBV) comprises Suvarna bhasma in an amount ranging from 3 wt % to 5 wt % of the total weight of SBV, Mouktik bhasma in an amount ranging from 23 wt % to 30 wt % of the total weight of SBV, Guduchi sattva in an amount ranging from 48 wt % to 54 wt % of the total weight of SBV, and at least one excipient in an amount ranging from 10 wt % to 25 wt % of the total weight of SBV along with binder as an excipient. The binder is selected from the group consisting of gum acacia, guar gum, and xanthan gum.

Typically, SBV contains 4 wt % Suvarna bhasma, 26 wt % Mouktik bhasma, and 53 wt % Guduchi Sattva which are bound together with a natural gum such as gum acacia which is in an amount of 17 wt %.

Suvarna Bhasmadi Vati (SBV) in solid dosage form is prepared by mixing Suvarna bhasma, Mouktik bhasma, Guduchi sattva, and at least one first excipient with water to obtain a dough. The Suvarna bhasma is obtained by triturating incinerated Suvarna with fresh juice of the leaves of Ocimum sanctum. The dough is pelletized to obtain pellets. Pellets having an average weight of 5 gm are formed from the dough and tray-dried at temperature in the range of 40° C. to 45° C. The dried pellets are ground in a mixer grinder to obtain a first mixture containing dry granules having powder to granule ratio of 30:70, which is then compressed to obtain Suvarna Bhasmadi Vati (SBV) in solid dosage form.

The average weight of the uncoated tablets is 240 mg±5%. The typical shelf life of the tablets is 3 years. The SBV is administered at a dose of 300 mg to 500 mg per day by oral administration.

The second container contains Mouktikyukta Kamdudha Vati (MKV) in solid dosage form, which is prepared from Mouktik bhasma in an amount ranging from 10 wt % to 14 wt % of the total weight of MKV, Shankha bhasma in an amount ranging from 10 wt % to 14 wt % of the total weight of MKV, Shouktik bhasma in an amount ranging from 10 wt % to 14 wt % of the total weight of MKV, Kapardik bhasma in an amount ranging from 10 wt % to 14 wt % of the total weight of MKV, Praval bhasma in an amount ranging from 10 wt % to 14 wt % of the total weight of MKV, Guduchi sattva in an amount ranging from 10 wt % to 14 wt % of the total weight of MKV, Shuddha Gairik in an amount ranging from 10 wt % to 14 wt % of the total weight of MKV, and at least one excipient in an amount ranging from 10 wt % to 30 wt % of the total weight of MKV. In an embodiment of the present disclosure, the excipient is a binder, selected from the group consisting of gum acacia, guar gum, and xanthan gum.

Mouktikyukta Kamdudha Vati (MKV) in solid dosage form is prepared by mixing Mouktik bhasma, Shankha bhasma, Shouktik bhasma, Kapardik bhasma, Praval bhasma, Guduchi sattva, Shuddha Gairik and at least one second excipient with water to obtain a dough. In one embodiment, for optimum effect, MKV is prepared from equal amounts (12 wt % each) of Mouktik Bhasma, Praval Bhasma, Shankha Bhasma, Shouktik Bhasma, Kapardik bhasma, Shudhha Gairik, and Guduchi Sattva. The dough is pelletized to obtain pellets which are dried at temperatures in the range of 40° C. to 45° C. The dried pellets are ground to obtain a first mixture having powder to granule ratio of 30:70 and compressed to obtain Mouktikyukta Kamdudha Vati (MKV) in solid dosage form. The average weight of each uncoated tablet is 300 mg±5%. The typical shelf life of the tablets is 3 years. MKV is administered at a dose of 800 mg to 1200 mg per day by oral administration.

The third container contains Trailokya chintamani Rasa Vati (TCR) in solid dosage form, which is prepared from Kajjali in an amount ranging from 0.012 wt % to 0.016 wt % of the total weight of TCR, Suvarna bhasma, Roupya bhasma, Heerak bhasma, Loha bhasma, Abhrak bhasma, Tamra bhasma, Shankha bhasma, Mouktik bhasma, Praval bhasma, Shuddha Hartaal and Shuddha Manahshila, each in an amount ranging from 0.005 wt % to 0.009 wt % of the total weight of TCR, Shuddha Kapardik in an amount ranging from 3.5 wt % to 3.9 wt % of the total weight of TCR, Rasasindoor in an amount ranging from 2 wt % to 6 wt % of the total weight of TCR, Vaikrant bhasma in an amount ranging from 0.75 wt % to 1.25 wt % of the total weight of TCR, Shuddha Tankan, Shuddha Vatsanabh dried root and Marich dried fruit powders, each in an amount ranging from 5.1 wt % to 7.1 wt % of the total weight of TCR, Suntha dried rhizome, Pippali dried inflorescence, Lavang dried buds, Jatiphal dried seed and Haritaki dried pericarp powders, each in an amount ranging from 1.4 wt % to 1.6 wt % of the total weight of TCR, all the extracts from Chitrak dried root, Shigru fresh root bark, Ardrak fresh rhizome and Nimbuk fresh fruit, together in an amount ranging from 53.5 wt % to 56.5 wt % of the total weight of TCR, and at least one excipient in an amount ranging from 5 wt % to 15 wt % of the total weight of TCR.

During preparation, intermittent trituration are performed with Arka dugdha (latex), Nirgundi fresh leaf juice, Surana fresh rhizome juice and Snuhi Kshir (latex), each in an amount ranging from 0.13% v/v to 0.17% v/v, Chitrak dried root decoction in an amount ranging from 10.5% v/v to 14.5% v/v, Shigru fresh root bark juice in an amount ranging from 3.2% v/v to 3.6% v/v, Ardrak fresh rhizome juice in an amount ranging from 30.75% v/v to 34.75% v/v and Nimbuk fresh fruit juice in an amount ranging from 48.75% v/v to 52.75% v/v of the total liquid media used for trituration, wherein Kajjali, Suvarna bhasma, Roupya bhasma, Heerak bhasma, Tamra bhasma, Abhrak bhasma, Loha bhasma, Mouktik bhasma, Shankha bhasma, Praval bhasma, Shuddha Hartaal, Shuddha Manahshila, Rasasindoor, Shuddha Tankan and Vaikrant bhasma powders, each has a particle size in the range of 53 to 73 microns; Shuddha Vatsanabh, Marich, Sunthi, Pippali, Lavang, Jatiphal and Haritaki powders, each has a particle size in the range of 180 to 250 microns and gum acacia powder has a particle size in the range of 150 to 180 microns.

Trailokya chintamani Rasa Vati (TCR) in solid dosage form is prepared by mixing and powdering Kajjali, Suvarna bhasma, Roupya bhasma, Heerak bhasma, Tamra bhasma, Abhrak bhasma, Loha bhasma, Mouktik bhasma, Shankha bhasma, Praval bhasma, Shuddha Hartaal and Shuddha Manahshila is to obtain a first powder. The first powder is sequentially triturated with a first liquid comprising decoction of fresh juice and/or latex of plants selected from the group consisting of Arkadugdha, Nirgundi, Surana, Snuhi Kshir, and Chitrak, to obtain a mixture. The powdered product is mixed with a second powder comprising Shuddha Kapardik and sealed with the paste of Shuddha Tankan and Arka dugdha, dried and placed in earthen vessels (casseroles) and sealed using clay. The sealed arrangement is incinerated at 700-750° C., allowed to cool and powdered. The second powdered product is mixed with a third powder comprising Rasasindoor and Vaikrant bhasma and ground for 2-4 hrs. The third mixture is triturated with a second liquid comprising decoction of Chitrak, fresh juice of Shigruin, fresh juice of Ardrak and fresh juice of Nimbuk to obtain a fourth mixture. The fourth mixture is dried at 45° C. and powdered to obtain a third powdered product. The third powdered product is mixed with a fourth powder comprising Shuddha Tankan, Shuddha Bachnag, Marich, Sunthi, Pippali, Lavang, Jayphal, and Haritaki, to obtain a fifth mixture. The fifth mixture is triturated with a third liquid comprising Nimbuk and Adrak to obtain a sixth mixture. The sixth mixture is dried at 45° C. and powdered to obtain a fourth powdered product. The fourth powdered product is mixed with at least one third excipient and water to form a dough. The dough is pelletized to obtain pellets which are dried at 45° C. The dried pellets are ground to obtain a first mixture having powder to granule ratio of 70:30 and compressed to obtain Trailokya chintamani Rasa Vati (TCR) in solid dosage form. In one embodiment, the tablets of TCR, have a weight of 137.5 mg with ±5% acceptable variation. The typical shelf life of these tablets is 5 years. TCR is administered at a dose of 200 to 300 mg per day by oral route.

Vitex negundo L. is also known as Nilanirgundi belong to family Lamiaceae. A large shrub or small tree with elegant palmate leaves, spikes of slender, fragrant mauve-coloured flowers and dark reddish brown to black fruits resembling pepper corns in shape and aroma, native to the Mediterranean region of Southern Europe and west Asia. It is cultivated in the gardens of India and throughout warm temperate and subtropical regions.

Amorphophallus paeniifolius (Dennst.) Nicolson (syn. A. campanulatus (Roxb.) Blume) is also known as Surana belong to family Araceae. A perennial herb with purple coloured flowers arranged on spadix (appearing before leaves) and with large, globose depressed underground corn. It is found almost throughout India and also cultivated.

Calotropis procera (Aiton) Dryand is also known as Adityapushpika/Alarka belong to family Asclepiadaceae. It is a spreading shrub or a small tree to 4 m, oozing copious milky sap when cut or broken. It is found almost throughout India however it is native to tropical Africa and Asia and introduced to the Southern United States and Brazil (Crothers and Newbound, 1998). It is naturalized in Australia, many Pacific islands, Mexico, Central and South America and the Caribbean islands.

Euphorbia neriifolia L. is also known as Gudha/Nagarika/Nanda/Nistrinsapatra/Patrasnuhi belong to family Euphorbiaceae. It is a prickly, large, branched, erect, succulent leafless shrub that occurs in dry, rocky and hilly areas of North, Central and South India.

Plumbago zeylanica(Linn.) is also known as Chitraka belong to family Plumbaginaceae. It is a large perennial undershrub, found throughout India in plains and occasionally grown in the gardens.

Moringa oleifera Lam. is also known as Shigru/Shevga belong to family Moringaceae. It is small deciduous tree. Indigenous to Sub-Himalayan tracts, widely cultivated throughout the tropical countries. It also naturalized in Africa and tropical America.

Citrus limon (Linn.) Osbeck is also known as Nimbu belong to family Rutaceae. A straggling bush or small tree, 3-4 m high with thorny branches, cultivated in many parts of the India and Southeast Asia.

Aconitum ferox Wall. Ex Ser. is known as Vatsanabha belong to family Ranunculaceae. A biennial herb with thick tuberous root, distributed in Himalayas from Kashmir to Nepal and Sikkim at an altitude of 2100-3800 m.

Piper nigrum (Linn.) is also known as Marica belong to family Piperaceae. A stout and woody perennial climber clinging to the support by means of adventitious roots throughout joints nodes; a glabrous aromatic plant growing well in heavy Konkan Southwards especially in heavy rainfall areas, indigenous to Western Ghats and Assam, now found abundantly cultivated in Konkan Southwards especially in North Canara and Kerala.

Zingiber officinale Rosc. is also known as Sunthi belong to family Zingiberaceae. It is a perennial aromatic stout horizontally growing rhizomatous herb having several sympodial lateral tubers and an elongated erect leafy shoot up to 60 cm high; cultivated in warmer parts of India without any reports of its natural occurrence in the wild.

Piper longum(Linn.) is known as Pippali belong to family Piperaceae. The plant is a slender climber. Distributed in warmer regions of the country i.e., Western Ghats, central Himalayas to Assam, Khasi and Miker hills and lower hills of Bengal.

Syzygium aromaticum(Linn.) Merril & Perry is known as Lavanga belong to family Myrtaceae. An evergreen tree 9-10 m high, sometimes grows taller, a native of Moluccas, cultivated in many parts of the world and also to a considerable extent in Southern India.

Myristica fragrans Houtt. is known as Jaiphal/nutmeg belong to family Myristicaceae. It is a tropical evergreen tree that reaches about 65 feet tall. It is native to Indonesia and cultivated widely in India for its fruit.

Terminalia chebula (Gaertn.) Retz. is known as Haritaki belong to family Combretaceae. Plant is found throughout India chiefly in deciduous forests. It occurs abundantly in North India. Its range extends southwards at 300 to 900 m altitude.

Acacia nilotica (Linn.) Willd. Ex Delile ssp. Indica (Benth.) Brenan (syn. A. Arabicaauct. Non Willd.) is known as Babbulah belong to family Mimosaceae. Drug consists of dried gummy exudate from the stem and branches. It is distributed throughout the warmer and dried parts of India.

All the plants used in TCR are obtained from Bharatiya Sanskriti Darshan Trust (BSDT), Wagholi, Pune. Other related species of each of the above plants also can be used in the herbo-mineral metallic pharmaceutical kit of the present disclosure.

The fourth container contains Kumari Kalpa Vati (KKV) in solid dosage form, which is prepared from Suntha dried rhizome, Marich dried fruit, Pippali dried inflorescence, Bhumiamalaki dried whole plant, Bibhitaki dried pericarp, Haritaki dried pericarp, Musta dried rhizome, Vidang dried fruit, Sharpunkha dried whole plant and Kutki dried root powders, each in an amount ranging from 6.5 wt % to 10.5 wt % of the total weight of KKV, dried Kumari leaf pulp in an amount ranging from 2.5 wt % to 5 wt % of the total weight of KKV, and at least one excipient in an amount ranging from 10 wt % to 20 wt % of the total weight of KKV. The powders of Suntha, Marich, Pippali, Bhumiamalaki, Bibhitaki, Haritaki, Musta, Vidang, Sharpunkha and Kutki each have a particle size in the range of 180 to 250 microns. In an embodiment, the excipient is gum acacia having a particle size in the range of 150 to 180 microns.

Zingiber officinale Rosc. is also known as Sunthi belong to family Zingiberaceae. It is a perennial aromatic stout horizontally growing rhizomatous herb having several sympodial lateral tubers and an elongated erect leafy shoot up to 60 cm high; cultivated in warmer parts of India without any reports of its natural occurrence in the wild.

Piper nigrum (Linn.) is also known as Marica belong to family Piperaceae. A stout and woody perennial climber clinging to the support by means of adventitious roots throughout joints nodes; a glabrous aromatic plant growing well in heavy Konkan Southwards especially in heavy rainfall areas, indigenous to Western Ghats and Assam, now found abundantly cultivated in Konkan Southwards especially in North Canara and Kerala.

Piper longum (Linn.) is known as Pippali belong to family Piperaceae. The plant is a slender climber. Distributed in warmer regions of the country i.e., Western Ghats, central Himalayas to Assam, Khasi and Miker hills and lower hills of Bengal.

Phyllanthus fraternus Webst. (syn. P. niruri auct. Pl. non (Linn.)) is known as Tamalaki belong to family Euphorbiaceae. An annual herb up to 60 cm high found in Central and Southern India extending to Sri Lanka. Common throughout the hotter parts of India in waste places and shady gardens.

Terminalia bellirica (Gaertn.) Roxb. (syn. T. punetata Roxb., Myrobalamus belerica B. Gaertn.) is known as Bibhiutaka belongs to the family Combretaceae. The plant is common throughout India in plains and lower hills, chiefly in deciduous forests, at 900 m elevation where the climate is not very dry. It is also found in forests of Burma and Sri Lanka.

Terminalia chebula (Gaertn.) Retz. is known as Haritaki belong to family Combretaceae. Plant is found throughout India chiefly in deciduous forests. It occurs abundantly in North India. Its range extends southwards at 300 to 900 m altitude.

Picrorhiza kurroa Royle ex Benth. is known as Katuka belong to family Scrophulariaceae. A small hairy herb with perenniating rhizomes growing in the hilly regions of the Northwest Himalayas in India, and Nepal at an altitude of 2700 to 4000 m.

Embelia ribes Burm. F. is known as Vidanga belong to family Myrsinaceae. It is a climbing, perennial shrub, found throughout India, ascending up to 1500 m in the hills.

Cyperus rotundus(Linn.) is known as Mustaka belong to family Cyperaceae. The plant is distributed throughout the plains of India up to 1800 m elevation. It grows in moist areas, rice fields and along water courses.

Tephrosia purpurea (L.) Pers. is known as sarapunkha belong to family Fabaceae. The plant is distributed throughout India and Malesia. It grows in dry grasses and roadsides.

Aloe vera (Linn.) Burm. f. is known as Kumari belong to family Liliaceae. A dwarf succulent, perennial herb producing dense, aggregated, fleshy leaves with horny prickles in the margins, distributed throughout India especially in hot and dry localities of North-Western India up to an altitude of 800 m.

Acacia nilotica (Linn.) Willd. Ex Delile ssp. Indica (Benth.) Brenan (syn. A. Arabica auct. Non Willd.) is known as Babbulah belong to family Mimosaceae. Drug consists of dried gummy exudate from the stem and branches It distributed throughout the warmer and dried parts of India.

All the plants used in KKV are obtained from Bharatiya Sanskriti Darshan Trust (BSDT), Wagholi, Pune. Other related species of each of the above plants also can be used in the herbo-mineral metallic pharmaceutical kit of the present disclosure.

Kumari Kalpa Vati (KKV) in solid dosage form is prepared by mixing a powdered mixture comprising Shunthi powder, Marich powder, Pippali powder, Bhumiamalaki powder, Bibhitaki powder, Haritaki powder, Musta powder, Vidang powder, Sharpunkha powder, Kutki powder, and at least one fourth excipient in a ratio of 5:1 to obtain a first mixture. The first mixture is mixed with a ground pulp comprising Kumari, in the ratio of 1:2 (w/w), to obtain a dough. The dough is pelletized to obtain pellets which are dried at 45° C. The dried pellets are ground to obtain a second mixture having powder to granule ratio of 70:30. The second mixture is compressed to obtain Kumari Kalpa Vati (KKV) in solid dosage form.

In an embodiment, the granules are compressed in a tablet punching machine to obtain compressed tablets of KKV weighing 300 mg each with ±5% acceptable variation. The typical shelf life of these tablets is 3 years. The KKV is administered at a dose of 1600 to 2400 mg per day by oral route.

The fifth container contains Khadirarishta (KHA) in liquid dosage form, which is prepared from Khadir dried stem bark and Devdaru dried heartwood coarse powders, each in an amount ranging from 8.75 wt % to 12.75 wt % of the total weight of KHA, Daruharidra dried stem coarse powder in an amount ranging from 2.3 wt % to 6.3 wt % of the total weight of KHA; Haritaki dried pericarp, Bibhitaki dried pericarp and Amalaki dried pericarp coarse powders, each in an amount ranging from 1 wt % to 1.5 wt % of the total weight of KHA, Jatiphal dried seed, Tamalpatra dried leaf, Bruhad Ela dried pods, Lavang dried bud, Dalchini dried stem bark, Kankol dried fruit, Nagkeshar dried stamens and Pippali dried inflorescence powders, each in an amount ranging from 0.19 wt % to 0.23 wt % of the total weight of KHA, jaggery crushed in an amount ranging from 60 wt % to 70 wt % of the total weight of KHA, Dhataki flowers in an amount ranging from 3.1 wt % to 3.5 wt % of the total weight of KHA. Each of Khadir, Devdaru, Daruharidra, Haritaki, Bibhitaki and Amalaki coarse powders have a particle size in the range of 4 to 8 mm and the fine powders of Jatiphal, Tamalpatra, Bruhad Ela, Lavang, Dalchini, Kankol, Nagkeshar and Pippali have a particle size in the range of 150 to 250 microns.

Khadirarishta (KHA) in liquid dosage form is prepared by soaking overnight in water a first powder comprising Khadir coarse powder, Devdaru coarse powder, Daruharidra coarse powder, Haritaki coarse powder, Bibhitaki coarse powder, Amalaki coarse powder, and heating to obtain a decoction. Typically, the mixture is boiled to prepare decoction by reducing it to ¼^{th}. The decoction is filtered through a muslin cloth to obtain a filtrate and cooled. Crushed jaggery is added to the filtrate and dissolved completely to obtain a slurry. The slurry is filtered and mixed with Dhataki flowers as natural inoculum and with a second fine powder comprising Jatiphal powder, Tamalpatra powder, Bruhad Ela powder, Lavang powder, Dalchini powder, Kankol powder, Nagkeshar powder, Pippali powder, to obtain a first mixture. The first mixture is incubated in a closed barrel at a temperature in the range of 32-34° C. for a period of 21 to 30 days to obtain a fermented mixture. The fermented mixture is filtered to obtain Khadirarishta (KHA) in liquid dosage form. Its typical shelf life of is 10 years.

Acacia catechu (Linn. f.) Willd is known as Khadira belong to family Mimosaceae. A moderate sized, deciduous spinous tree, growing in dry exposed forests throughout warmer regions in India ascending to 1500 m altitude.

Cedrus deodara (Roxb. Ex D. Don) G. Don is known as Devadaru belong to family Pinaceae. A large evergreen coniferous tree reaching 40 to 50 m in height sometimes up to 60 m and up to 3 m in diameter with a conical crown and drooping branchlets. The plant is native to the western Himalayas from Kashmir to Uttarakhand and Nepal up to an altitude of 1200 to 3000 m.

Berberis aristata DC. Var. aristata is known as Daru haridra belong to family Berberidaceae. It is an erect, spinous, deciduous shrub usually 1.8 to 3.6 m high, found in Himalayas at an altitude of 1000-3000 m and in the Nilgiri hills in South India, altitude, 1000-2400 m.

Woodfordia fruticosa (L.) Kurz is known as Dhataki pusp belong to family Lythraceae. It is deciduous shrub, small in size but very conspicuous on dry, rocky hillsides. It found in Sri Lanka, South Konkan and on the Ghats and ascends the Himalayas up to 200-1800 m, but is rarer in South India.

Piper cubeba(Linn.) F. is known as Kankola belong to family Piperaceae. A woody perennial climber, indigenous to Indonesia and cultivated in India, Sri Lanka and Caribbean Islands as a spice.

Myristica fragrans Houtt is known as Jaiphal/nutmeg belong to family Myristicaceae. It is a tropical evergreen tree that reaches about 65 feet tall. It native to Indonesia and cultivated widely in India for its fruit.

Syzygium aromaticum (Linn.) Merril & Perry is known as Lavanga belong to family Myrtaceae. An evergreen tree 9-10 m high, sometimes grows taller, a native of Moluccas, cultivated in many parts of the world and also to a considerable extent in Southern India.

Mesua ferrea L. is known as Nagkesar belong to family Clusiaceae. It is evergreen tree often planted as an ornamental for its fragrant white flowers that yield a perfume; source of very heavy hardwood used for railroad ties. The plant is native of India and Sri Lanka.

Cinnamomum zeylanicum Nees is known as Darusita belong to family Lauraceae. The plant is native of India and Sri Lanka.

Cinnamomum tamala (Buch. Ham.) Nees & Eberm. is known as Tamalapatra belong to family Lauraceae. A small evergreen, moderate sized tree growing in sub-Himalayan tracts from Jammu eastwards to Bhutan between altitude 900-2300 m and in Khasi hills, altitude 900-1200 m.

Piper longum (Linn.) is known as Pippali belong to family Piperaceae. The plant is a slender climber. Distributed in warmer regions of the country i.e., Western Ghats, central Himalayas to Assam, Khasi and Miker hills and lower hills of Bengal.

Amomum subulatum Roxb. is also known as Sthulaila belongs to family Zingiberaceae. It is an evergreen herb with buds encaeastern sed in tight red bracts appearing in spring from the base of the red coloured rhizomes; native to Darjeeling, Sikkim and North East India.

All the plants used in preparing KHA are obtained from Bharatiya Sanskriti Darshan Trust (BSDT), Wagholi, Pune. Other related species of each of the above plants also can be used in the herbo-mineral metallic pharmaceutical kit of the present disclosure. The KHA is administered at a dose of 10 to 30 mL per day by oral route.

The compositions of SBV, MKV, TCR and KKV are prepared in the form of a solid dosage form selected from the group consisting of tablet, pill, and capsule. The composition of KHA is prepared in liquid dosage form comprising at least one liquid selected from the group consisting of water, alcohol and hydro alcohol.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure is further described in light of the following experiments which are set forth for illustration purpose only and not to be construed for limiting the scope of the disclosure. The following experiments can be scaled up to industrial/commercial scale and the results obtained can be extrapolated to industrial scale.

### EXPERIMENTAL DETAIL

### Example 1: Herbo-Mineral Metallic Pharmaceutical Kit

A herbo-mineral metallic pharmaceutical kit was prepared in accordance with the present disclosure. The components of the herbo-mineral metallic pharmaceutical kit are given in Table 1. It was found that the best results were obtained when the components were administered in the dosage form and frequency as provided in Table 1.

TABLE 1
Components of the herbo-mineral metallic pharmaceutical kit

| Sr. | | Dosage range | Dosage | Frequency of |
|---|---|---|---|---|
| No. | Components | per day | administered | dose |
| 1 | Suvarna | 300 mg to | 2 tablets of | 2 tablets in the |
| | Bhasmadi Vati | 500 mg | 197.5 mg (once) | morning after |
| | (SBV) | | | breakfast (8.00AM) |
| 2 | Mouktikyukta | 800 mg to | 2 tablets of | 1 tablet in the |
| | Kamdudha Vati | 1200 mg | 250 mg (twice) | morning after |
| | (MKV) | | | breakfast (8.00AM) - |
| | | | | 1 tablet in the |
| | | | | evening after |
| | | | | snacks (5.00PM) |
| 3 | Trailokyachintam | 200 to | 2 tablets of | 2 tablets in the |
| | ani Rasa (TCR) | 300 mg | 125 mg (once) | evening after |
| | | | | snacks (5.00PM) |
| 4 | Kumari Kalpa | 1600 to | 4 tablets of | 2 tablets after |
| | Vati (KKV) | 2400 mg | 250 mg (twice) | lunch and 2 tablets |
| | | | | after dinner |
| 5 | Khadirarishta | 10 to | 10 ml (twice) | After Lunch and |
| | (KHA) | 30 mL | | Dinner |

### Experiment 1: Suvarna Bhasmadi Vati (SBV) in Accordance with the Present Disclosure

Suvarna Bhasmadi Vati (SBV) was prepared by using the following ingredients as given in Table 2.

TABLE 2
Ingredients used for preparing SBV

| | | | Quantity for | Quantity for | Quantity for |
|---|---|---|---|---|---|
| Sr. | | Latin name/ | 5 kg batch- | 5 kg batch- | 5 kg batch- |
| No. | Contents | English Name | Batch 1 | Batch 2 | Batch 3 |
| 1 | Suvarna bhasma | Incinerated Gold | 210.97 g | 110 g | 350 g |
| | | | (4.2%) | (2.2%) | (7%) |
| 2 | Mouktik bhasma | Incinerated Pearl | 1318.56 g | 1700 g | 1000 g |
| | | | (26.37%) | (34%) | (20%) |
| 3 | Guduchi sattva (aqueous | Starch of | 2637.1 g | 2940 g | 2250 g |
| | extract of Tinospora | Tinospora | (52.74%) | (58.8%) | (45%) |
| | cordifolia/ | cordifolia/ | | | |
| | sinensis) | sinensis | | | |
| 4 | Gum Acacia powder | | 833.33 g | 250 g | 1400 g |
| | | | (16.66%) | (5%) | (28%) |

### Process for Preparing Suvarna Bhasmadi Vati (SBV):

### Step 1: Preparation of Suvarna Bhasma

Initially, 300 gm of Suvarna foils were amalgamated with 2500 gm of metallic mercury and 2500 gm of sulphur powder having particle size 150 microns and then incinerated 18 times each at 650° C. for 6 hours to get incinerated Suvarna. For stabilization, the incinerated Suvarna was triturated for 6 hours with 200 ml fresh juice of the leaves of Ocimum sanctum (Tulsi) and further incinerated at 600° C. for 5 hours. This process of incineration in Tulsi juice was repeated 25 times to obtain Suvarna bhasma having particle size in the range of 20-500 nm (average particle size 350 nm). The particle size of the so obtained Suvarna bhasma were analysed by NanoBrook particle size analyser.

Ocimum sanctum (Tulsi) is also known as holy basil of the family Lamiaceae. Ocimum sanctum was obtained from Bharatiya Sanskriti Darshan Trust (BSDT), Wagholi, Pune.

### Specification of Suvarna Bhasma:

Description: Brown coloured, very fine free flowing powder

Loss on Drying—NMT 0.5% w/w

Loss on Ignition—NMT 1% w/w

Acid insoluble ash—90 to 98% w/w

Assay as Au—NLT 85% w/w

Step 2: Preparation of Mouktik bhasma

8 kg Mountie (pearl) was boiled in 32 L of butter milk having curd to water ratio of 1:2 w/v and pH of 3, to obtain purified Mouktik. The so obtained purified Mouktik was powdered and triturated with 4 L of rose water to obtain triturated powder. The triturated powder was then incinerated using cow-dung cakes at 700° C. to obtain Mouktik bhasma (incinerated pearl).

Specification of Mouktik bhasma:

Description: Greyish white coloured, very fine powder

Loss on Drying—NMT 0.5% w/w

Acid insoluble ash—NMT 2% w/w

Calcium assay—38 to 40% w/w

pH-10 to 11

Step 3: Preparation of Guduchi Sattva—by Extraction of Starch from Tinospora cordifolia

50 Kg of fresh stems of Tinospora cordifolia were chopped into small pieces. These pieces were crushed and then soaked for 12 hours in 4 times (w/v) of potable water (200 L) in a stainless steel vessel. The mixture was macerated in water thoroughly and filtered slowly to obtain solution containing aqueous extract of Tinospora cordifolia. The solution thus obtained was kept aside for 12 hours to obtain supernatant and smooth starchy sediment of Tinospora cordifolia. The supernatant was carefully separated to obtain smooth starchy sediment. The smooth starchy sediment of Tinospora cordifolia was evaporated in an oven at 45° C. to obtain Guduchi Sattva in the form of dry starch.

Specification of Starch Extract of Tinospora cordifolia:

Description: Greyish white coloured very fine free flowing starchy powder

Loss on Drying—NMT 5% w/w

Acid insoluble ash—NMT 1% w/w

Gelation temperature—60 to 75° C.

### Step 4: Preparation of SBV

In a mixer, Suvarna bhasma, Mauktik bhasma, Guduchi Sattva (starch from Tinospora cordifolia), and gum acacia were mixed in the proportion given in Table 2. To this mixture, sufficient amount of water was added to obtain a dough. The dough was further pelletized to obtain pellets. The pellets were dried in oven at 45° C. to obtain dried pellets. The dried pellets were grinded to obtain granules having powder to granule ratio of 30:70. The mixture of granules and powder was compressed in tablet punching machine to obtain compressed tablet of weight 237±5 mg.

### Specifications of SBV:

Appearance: Grey colour, round

Shape: Biconvex tablets

Weight variation: 0.2210 to 0.2300

Average weight: 0.2300 to 0.2500

Hardness: 1-2 Kg/cm²

Friability: NMT 1% w/w

Disintegration Time: NMT 30 min

Diameter: 7 to 7.5 mm

Width: 3.3 to 4.6 mm

Acute toxicity: LD 50>2000 mg/kg

### Experiment 2: Mouktikyukta Kamdudha Vati (MKV) in Accordance with the Present Disclosure

Mouktikyukta Kamdudha Vati (MKV) was prepared by using the following ingredients as given in Table 3.

TABLE 3
Ingredients used for preparing Mouktikyukta Kamdudha Vati (MKV)

| | | | Quantity for | Quantity for | Quantity for |
|---|---|---|---|---|---|
| | | | 30 kg | 30 kg | 30 kg |
| Sr. | | Latin name/ | batch- | batch- | batch- |
| No. | Contents | English Name | Batch 1 | Batch 2 | Batch 3 |
| 1 | Mouktik bhasma | Incinerated Pearl | 3572 g | 3500 | 4000 |
| | | | (11.7%) | (11.5%) | (13.2%) |
| 2 | Shankha bhasma | Incinerated Conch | 3572 g | 3000 | 3800 |
| | | | (11.7%) | (9.9%) | (12.5%) |
| 3 | Shouktik bhasma | Incinerated Pearl | 3572 g | 3000 | 3800 |
| | | Shell | (11.7%) | (11.5%) | (12.5%) |
| 4 | Kapardik bhasma | Incinerated Cowrie | 3572 g | 3000 | 3800 |
| | | | (11.7%) | (9.9%) | (12.5%) |
| 5 | Praval bhasma | Incinerated Coral | 3572 g | 3500 | 3800 |
| | | | (11.7%) | (11.5%) | (12.5%) |
| 6 | Guduchi | Starch of | 3572 g | 3500 | 4000 |
| | battva(aqueous | Tinospora | (11.7%) | (11.5%) | (13.2%) |
| | extract of | cordifolia/ | | | |
| | Tinospora | sinensis | | | |
| | cordifolia/ | | | | |
| | sinensis) | | | | |
| 7 | ShuddhaGairik | Red ochre roasted | 3572 g | 3000 | 3800 |
| | | in cow ghee | (11.7%) | (9.9%) | (12.5%) |
| 8 | Gum Acacia powder | | 5000 g | 7500 | 3000 |
| | | | (16.5%) | (24.7%) | (12.5%) |

### Process for Preparing Mouktikyukta Kamdudha Vati (MKV):

All the five bhasma (Mouktik bhasma, Shankha bhasma, Shouktik bhasma, Kapardik bhasma, Praval bhasma) are prepared using textual methods, Guduchi Sattva (starch of Tinospora cordifolia/sinensis) and Shudhha Gairikand gum acacia powder were mixed in a proportion given in Table 3. To this mixture, sufficient amount of potable water was added to obtain a dough. The dough was further pelletized to obtain pellets. The pellets were dried in oven at 45° C. to obtain dried pellets. The dried pellets were grinded to obtain granules having powder to granule ratio of 30:70. The mixture of granules and powder was compressed in tablet punching machine to obtain compressed tablet of weight 300±5 mg.

### Specification of Shankha Bhasma:

Description—Greyish white very fine powder

Loss on drying—NMT 1% w/w

Acid insoluble ash—NMT 2% w/w

pH—9 to 10

Calcium assay as Ca—38 to 40% w/w

### Specification of Shouktik Bhasma:

Description—Greyish white very fine powder

Loss on drying—NMT 1% w/w

Acid insoluble ash—NMT 2% w/w

pH—10 to 11

Calcium assay as Ca—38 to 40% w/w

### Specification of Kapardik Bhasma:

Description—Greyish white very fine powder

Loss on drying—NMT 1% w/w

Acid insoluble ash—NMT 2% w/w

pH—10 to 11

Calcium assay as Ca—38 to 40% w/w

### Specification of Praval Bhasma:

Description—Greyish white very fine powder

Loss on drying—NMT 1% w/w

Acid insoluble ash—NMT 2% w/w

pH—10 to 11

Calcium assay as Ca—40 to 45% w/w

### Specification of Mouktik (Pearl) Bhasma:

Description—Greyish white very fine powder

Loss on drying—NMT 1% w/w

Acid insoluble ash—NMT 2% w/w

pH—10 to 11

Calcium assay as Ca—38 to 40% w/w

### Specification of ShudhhaGairik (Red Ochre):

Loss on drying—NMT 1% w/w

Iron assay—NLT 15% w/w

Silica assay—18 to 20% w/w

Oil content—3 to 3.5% w/w

Specification of Guduchi Sattva (Starch of Tinospora cordifolia/Sinensis):

Loss on drying—4 to 5% w/w

Acid Insoluble ash—NMT1% w/w

Gelation temperature—60 to 75° C.

### Specification of MKV:

Description: Light brown colour

Shape: Round biconvex tablet

Weight variation: 0.2850 to 0.3150

Average weight: 0.2900 to 0.3100

Hardness: 2 to 4 Kg/cm²

Friability: NMT 1% w/w

Disintegration Time: NMT 30 min

Diameter: 7 to 8 mm

Width: 3 to 4 mm

Acute Toxicity: LD 50>2000 mg/kg

### Experiment 3: Trailokya Chintamani Rasa Vati (TCR) in Accordance with the Present Disclosure

Trailokya chintamani Rasa Vati (TCR) was prepared by using the following ingredients as given in Table 4.

TABLE 4
Ingredients used for preparing Trailokyachintamani Rasa Vati (TCR)

| | | | | | Qty. of | Qty. of |
|---|---|---|---|---|---|---|
| | | | | Qty. of raw | raw | raw |
| | | | | materials | materials | materials |
| | | | Form used | used in | used in | used in |
| S. | | | (Particle | 4.95 Kg- | 4.95 kg- | 4.95 kg- |
| No. | Ingredient | Part used | size-μm) | Batch 1 | Batch 2 | Batch 3 |
| | First Powder: | | | | | |
| | Step 1 | | | | | |
| 1 | Kajjali | Triturated | Powder | 0.666 g | 0.466 g | 0.766 g |
| | | | (53-75) | (0.32%) | (0.22%) | (0.37%) |
| 2 | Suvarna Bhasma | Incinerated | | 0.333 g | 0.233 g | 0.383 g |
| | | | | (0.161%) | (0.10%) | (0.18%) |
| 3 | Roupya Bhasma | | | 0.333 g | 0.233 g | 0.383 g |
| | | | | (0.161%) | (0.10%) | (0.18%) |
| 4 | Heerak Bhasma | | | 0.333 g | 0.233 g | 0.383 g |
| | | | | (0.161%) | (0.10%) | (0.18%) |
| 5 | Tamra Bhasma | | | 0.333 g | 0.233 g | 0.383 g |
| | | | | (0.161%) | (0.10%) | (0.18%) |
| 6 | Abhrak Bhasma | | | 0.333 g | 0.233 g | 0.383 g |
| | | | | (0.161%) | (0.10%) | (0.18%) |
| 7 | Loha Bhasma | | | 0.333 g | 0.233 g | 0.383 g |
| | | | | (0.161%) | (0.10%) | (0.18%) |
| 8 | Mouktik Bhasma | | | 0.333 g | 0.233 g | 0.383 g |
| | | | | (0.161%) | (0.10%) | (0.18%) |
| 9 | Shankha Bhasma | | | 0.333 g | 0.233 g | 0.383 g |
| | | | | (0.161%) | (0.10%) | (0.18%) |
| 10 | Praval Bhasma | | | 0.333 g | 0.233 g | 0.383 g |
| | | | | (0.161%) | (0.10%) | (0.18%) |
| 11 | Shuddha Hartaal | Triturated | | 0.333 g | 0.233 g | 0.383 g |
| | | | | (0.161%) | (0.10%) | (0.18%) |
| 12 | Shuddha | | | 0.333 g | 0.233 g | 0.383 g |
| | Manahshila | | | (0.161%) | (0.10%) | (0.18%) |
| | First Liquid: | | | | | |
| | Group 1 | | | | | |
| 13 | Arka dugdha | Latex | Liquid | 55 ml | 35 ml | 65 ml |
| 14 | Chitrak | Root | Decoction | 125 ml | 85 ml | 140 ml |
| 15 | Nirgundi | Leaf | Fresh juice | 55 ml | 35 ml | 65 ml |
| 16 | Suran | Tuber | Fresh juice | 55 ml | 35 ml | 65 ml |
| 17 | Snuhi Kshir | Latex | Liquid | 55 ml | 35 ml | 65 ml |
| | Weight after | | | 210 g | 150 g | 240 g |
| | Step I | | | | | |
| | Second Powder: | | | | | |
| | Step II | | | | | |
| 18 | Shuddha Kapardik | Purified | Whole | 180 g | 130 g | 200 g |
| | | | | (3.63%) | (2.62%) | (4.04%) |
| | Weight after Step | | | 200 g | 150 g | 220 g |
| | II | | | (4.04%) | (3.03%) | (4.44%) |
| | Third Powder: | | | | | |
| | Step III | | | | | |
| 19 | Rasasindoor | Sublimated | Powder | 200 g | 150 g | 220 g |
| | | | (53-75) | (4.04%) | (3.03%) | (4.44%) |
| 20 | Vaikrant Bhasma | Incinerated | | 50 g | 37 g | 55 g |
| | | | | (1.01%) | (0.74%) | (1.11%) |
| | Second Liquid: | | | | | |
| | Group 2 | | | | | |
| 21 | Chitrak | Root | Decoction | 4.2 L | 3 L | 4.8 L |
| 22 | Shigru | Root bark | Fresh juice | 1.4 L | 0.98 L | 1.6 L |
| 23 | Ardrak | Tuber | Fresh juice | 2.2 L | 1.5 L | 2.4 L |
| 24 | Nimbuk | Fruit | fresh juice | 4.2 L | 3 L | 5 L |
| | Weight after Step | | | 1.2 kg | 0.85 kg | 1.4 kg |
| | III | | | | | |
| | Fourth Powder: | | | | | |
| | Step IV | | | | | |
| 25 | Shuddha Tankan | Dehydrated | Powder | 300 g | 212 g | 350 g |
| | | | (53-75) | (6.06%) | (4.28%) | (7.07%) |
| 26 | Shuddha Bachnag | Purified | Powder | 300 g | 212 g | 350 g |
| | | rhizome | (180-250) | (6.06%) | (4.28%) | (7.07%) |
| 27 | Marich | Fruit | | 300 g | 212 g | 350 g |
| | | | | (6.06%) | (4.28%) | (7.07%) |
| 28 | Sunthi | Dry rhizome | | 75 g | 53 g | 87 g |
| | | | | (1.5%) | (1.07%) | (1.75%) |
| 29 | Pippali | Inflorescence | | 75 g | 53 g | 87 g |
| | | | | (1.5%) | (1.07%) | (1.75%) |
| 30 | Lavang | Bud | | 75 g | 53 g | 87 g |
| | | | | (1.5%) | (1.07%) | (1.75%) |
| 31 | Jayphal | Nut | | 75 g | 53 g | 87 g |
| | | | | (1.5%) | (1.07%) | (1.75%) |
| 32 | Haritaki | Pericarp | | 75 g | 53 g | 87 g |
| | | | | (1.5%) | (1.07%) | (1.75%) |
| | Third Liquid: | | | | | |
| | Group 3 | | | | | |
| 33 | Nimbuk | Fruit | Fresh juice | 15 L | 10.5 L | 17 L |
| 34 | Ardrak | Tuber | Fresh juice | 10 L | 7 L | 12 L |
| | Weight after Step | | | 4.5 kg | 3.15 kg | 4.8 kg |
| | IV | | | | | |
| | Excipient: | | | | | |
| | Step V | | | | | |
| 35 | Gum acacia | Exudate | Powder | 450 g | 1800 g | 150 g |
| | (Excipient) | | (150-180) | (9.09%) | (36.36%) | (3.03%) |

### Process for Preparing Trailokyachintamani Rasa Vati (TCR):

Step I: Trituration of Metals and Mineral Drugs with First Liquid
i. The ingredients 1 to 12 (Table 4) were mixed properly in a stone mortar with pestle for 3 hr.
ii. This mixture was triturated sequentially with a first liquid containing decoction/fresh juice/latex of ingredients 13 to 17 (Table 4); 3, 7, 3, 3 and 3 times, respectively.
iii. The triturated mixture was dried in oven at 45° C. and then powdered fine to pass through mesh no. 80.

### Step II: Filling the Product of Step I in Purified Cowrie, Concealing and Incineration

iv. The powder obtained in Step I was filled in ingredient no. 18 (Table 4) and sealed with the paste prepared out of ingredient nos. 13 and 25 (Table 4). They were dried overnight at room temperature.
v. The dried material was then placed in earthen vessels (casseroles). Then the earthen vessel was covered with another earthen vessel and sealed using clay.
vi. The arrangement was incinerated at 700-750° C. The material was then removed after self-cooling and powdered to pass through mesh no. 200.
   Step III: Trituration of Product of Step II with Second Liquid
vii. The mixture obtained in Step II was mixed with ingredients 19 and 20 (Table 4) and grinded in a mortar with pestle for 3 hr.
viii. The mixture was sequentially triturated with a second liquid containing decoction/fresh juice of plant ingredients 21 to 24 (Table 4), for 21, 7, 7 and 7 times, respectively.
ix. The mixture obtained after last trituration was dried in oven at 45° C. and powdered to pass through mesh no. 80.
   Step IV: Trituration of Product of Step III with Third Liquid
x. The mixture obtained in Step II was mixed with ingredients 25 to 32 (Table 4).
xi. The mixture was sequentially triturated with a third liquid containing fresh juice of plant ingredients 33 and 34 (Table 4) for 7 times each.
xii. The mixture obtained after last trituration was dried in oven at 45° C. and powdered to pass through mesh no. 80.

### Step V: Tableting of Final Product Obtained in Step IV

xiii. The final mixture obtained in Step III was mixed with specific amount of gum acacia powder per batch (Table 4) and sufficient quantity of potable water was added to make a dough.
xiv. Small pellets were made out of this dough and dried in oven at 45° C.
xv. The dried pellets were granulated to form granules having granule to powder ratio of 70:30
xvi. The obtained granules were compressed into tablets of TCR, having weight of 137.5 mg with ±5% acceptable variation.

### Specification of TCR:

The detailed specification of TCR is provided in Table 5 below.

### Description:

Dark reddish brown coloured circular biconvex tablets having characteristic odour.

TABLE 5
Specification of TCR

| Parameter | Units | Specifications |
|---|---|---|
| Loss on drying at 105° C. | % w/w | 10-14 |
| Gold as Au | % w/w | 0.0130-0.0170 |
| Silver as Ag | % w/w | 0.0040-0.0080 |
| Copper as Cu | % w/w | 0.0090-0.0120 |
| Iron as Fe | % w/w | 0.1550-0.1750 |
| Calcium as Ca | % w/w | 0.4600-0.6600 |
| Potassium as K | % w/w | 1.4100-1.8100 |
| Sodium as Na | % w/w | 0.2300-0.6300 |
| Boron as B | % w/w | 1.1050-1.1450 |
| Arsenic as As | ppm | Less than 1 |
| Silicon as Si | % w/w | 0.5800-0.9800 |
| Aluminium as Al | % w/w | 0.0250-0.0650 |
| Magnesium as Mg | % w/w | 0.1050-0.4250 |
| Sulphur as S | % w/w | 0.3700-0.4100 |
| Mercury as Hg | % w/w | 2.0300-2.4300 |
| Cadmium as Cd | ppm | Less than 0.3 |
| Lead as Pb | ppm | Less than 10 |
| Micro- TVC | CFU/g | Less than 10⁵ |
| Micro- Y&M | CFU/g | Less than 10³ |
| E. coli | CFU/g | Absent |
| Aflatoxins | ppm | Absent |
| Tablet weight variation | mg | 130-145 |
| Tablet average weight | mg | 135 |
| Tablet average diameter | mm | 6.0 |
| Tablet average height | mm | 4.2 |
| Tablet hardness | kg/cm² | 1.5 to 2 |
| Tablet friability | % w/w | Not more than 1 |
| Tablet disintegration time | min | 15-20 |
| Acute toxicity | LD 50 | More than 2000 mg/ |
| | | kg body weight |

### Experiment 4: Kumari Kalpa Vati (KKV) in Accordance with the Present Disclosure

Kumari Kalpa Vati (KKV) was prepared using the following ingredients as given in Table 6.

TABLE 6
Ingredients used for preparing Kumari Kalpa Vati (KKV)

| | | | | Quantity | Quantity | Quantity |
|---|---|---|---|---|---|---|
| | | | Form used | for 30 kg | for 30 kg | for 30 kg |
| Sr. | | | (Particle | batch- | batch- | batch- |
| No. | Contents | Part used | size in μm) | Batch 1 | Batch 2 | Batch 3 |
| 1 | Sunthi | Dry rhizome | Powder | 2.5 kg | 2.3 kg | 2.7 kg |
| | | | (150-250) | (8.25%) | (7.59%) | (8.91%) |
| 2 | Marich | Fruit | | 2.5 kg | 2.3 kg | 2.7 kg |
| | | | | (8.25%) | (7.59%) | (8.91%) |
| 3 | Pippali | Inflorescence | | 2.5 kg | 2.3 kg | 2.7 kg |
| | | | | (8.25%) | (7.59%) | (8.91%) |
| 4 | Bhumiamalaki | Whole | | 2.5 kg | 2.3 kg | 2.7 kg |
| | | | | (8.25%) | (7.59%) | (8.91%) |
| 5 | Bibhitaki | Pericarp | | 2.5 kg | 2.3 kg | 2.7 kg |
| | | | | (8.25%) | (7.59%) | (8.91%) |
| 6 | Haritaki | Pericarp | | 2.5 kg | 2.3 kg | 2.7 kg |
| | | | | (8.25%) | (7.59%) | (8.91%) |
| 7 | Musta | Rhizome | | 2.5 kg | 2.3 kg | 2.7 kg |
| | | | | (8.25%) | (7.59%) | (8.91%) |
| 8 | Vidang | Fruit | | 2.5 kg | 2.3 kg | 2.7 kg |
| | | | | (8.25%) | (7.59%) | (8.91%) |
| 9 | Sharpunkha | Whole | | 2.5 kg | 2.3 kg | 2.7 kg |
| | | | | (8.25%) | (7.59%) | (8.91%) |
| 10 | Kutki | Whole | | 2.5 kg | 2.3 kg | 2.7 kg |
| | | | | (8.25%) | (7.59%) | (8.91%) |
| 11 | Kumari | Leaf | Pulp (Passed | 50 kg | 46 kg | 54 kg |
| | | | through-710) | | | |
| 12 | Gum Acacia | Exudate | Powder | 5 kg | 7 kg | 3 kg |
| | | | (150-180) | | | |

### Process for Preparing Kumari Kalpa Vati (KKV):

In a mass mixer, powders of all the ingredients from 1 to 10 and gum acacia were mixed in a proportion given in Table 6. To this mixture, double quantity (w/w) of grinded aloe vera pulp was added to obtain a dough. The dough was further pelletized to obtain pellets. The pellets were dried in oven at 45° C. to obtain dried pellets. There was no appreciable weight gain. The dried pellets were grinded to obtain granules having granule to powder ratio of 70:30. The granules and powder were compressed in tablet punching machine to obtain compressed tablets of KKV weighing 300 mg each with ±5% acceptable variation.

### Specification of Kumari Kalpa Vati (KKV):

Description: Light brown coloured tablets

Shape: Round biconvex

Weight variation: 0.285 to 0.315 gm

Average weight: 0.3 gm

Hardness: 2 to 4 Kg/cm²

Friability: Not More Than 1% w/w

Disintegration Time: 15-30 min

Diameter: 8 to 9 mm

Width: 4 to 5 mm

Heavy metal content: Within permissible limits

Microbial load: Within permissible limits

### Experiment 5: Khadirarishta (KHA) in Accordance with the Present Disclosure

Khadirarishta (KHA) was prepared by fermentation process using the following ingredients as given in Table 7.

TABLE 7
Ingredients used for preparing Khadirarishta (KHA)

| | | | | Quantity | Quantity | Quantity |
|---|---|---|---|---|---|---|
| | | | | for 50 L | for 50 L | for 50 L |
| | | | Form | finished | finished | finished |
| | | | used | product | product | product |
| Sr. | | | (Particle | (KHA)- | (KHA)- | (KHA)- |
| No. | Contents | Part used | size) | Batch 1 | Batch 2 | Batch 3 |
| 1 | Khadir | Bark | Coarse | 4.12 kg | 2.9 kg | 5.4 kg |
| | | | powder | (8.24%) | (5.8%) | (10.8%) |
| 2 | Devdaru | Heartwood | (4-8 | 4.12 kg | 2.9 kg | 5.4 kg |
| | | | mm) | (8.24%) | (5.8%) | (10.8%) |
| 3 | Daruharidra | Stem | | 1.65 kg | 1.15 kg | 1.7 kg |
| | | | | (3.3%) | (2.3%) | (3.4%) |
| 4 | Haritaki | Pericarp | | 0.55 kg | 0.39 kg | 0.76 kg |
| | | | | (1.1%) | (0.78%) | (1.52%) |
| 5 | Bibhitaki | Pericarp | | 0.55 kg | 0.39 kg | 0.76 kg |
| | | | | (1.1%) | (0.78%) | (1.52%) |
| 6 | Amalaki | Pericarp | | 0.55 kg | 0.39 kg | 0.76 kg |
| | | | | (1.1%) | (0.78%) | (1.52%) |
| 7 | Water | | Potable | 188 L | 188 L | 188 L |
| 8 | Gul | | Lumps | 25 kg | 25 kg | 25 kg |
| | | | | (50%) | (50%) | (50%) |
| 9 | Dhataki | Flower | Whole | 1.25 kg | 1.25 kg | 1.25 kg |
| | | | | (2.5%) | (2.5%) | (2.5%) |
| 10 | Jatiphal | Nut | Fine | 0.08 kg | 0.056 kg | 0.10 kg |
| | | | powder | (0.16%) | (0.112%) | (0.02%) |
| 11 | Tamalpatra | Leaf | (150- | 0.08 kg | 0.056 kg | 0.10 kg |
| | | | 250 μm) | (0.16%) | (0.112%) | (0.02%) |
| 12 | Badi Velchi | Fruit | | 0.08 kg | 0.056 kg | 0.10 kg |
| | | | | (0.16%) | (0.112%) | (0.02%) |
| 13 | Lavang | Bud | | 0.08 kg | 0.056 kg | 0.10 kg |
| | | | | (0.16%) | (0.112%) | (0.02%) |
| 14 | Dalchini | Bark | | 0.08 kg | 0.056 kg | 0.10 kg |
| | | | | (0.16%) | (0.112%) | (0.02%) |
| 15 | Kankol | Fruit | | 0.08 kg | 0.056 kg | 0.10 kg |
| | | | | (0.16%) | (0.112%) | (0.02%) |
| 16 | Nagkeshar | Stamens | | 0.08 kg | 0.056 kg | 0.10 kg |
| | | | | (0.16%) | (0.112%) | (0.02%) |
| 17 | Pippali | Inflores- | | 0.08 kg | 0.056 kg | 0.10 kg |
| | | cence | | (0.16%) | (0.112%) | (0.02%) |

### Process for Preparing Khadirarishta (KHA):

The ingredients from 1 to 6 (Table 7) were soaked overnight in 188 L of potable water. It was heated at 100° C. to prepare decoction by reducing it to 47 L. The decoction was filtered through a muslin cloth and allowed to self-cool. Ingredient no. 8 (Table 7) was added to this cooled decoction and dissolved completely to form a slurry. The slurry was filtered through mesh no. 32. Finally, ingredient no. 9 as natural inoculum and ingredient nos. 10 to 17 (Table 7) were added to the slurry and filled in a wooden barrel of 100 L capacity. The barrel was closed tightly and kept in a chamber having room temperature between 32-34° C. for a period of 21 to 30 days to complete the natural fermentation as confirmed by Candle Test. After assessing the completion of fermentation process, the finished product (KHA) was filtered through clean muslin cloth and stored in amber coloured bottles.

### Specification of Khadirarishta (KHA):

Description: Reddish brown hazy liquid, miscible in water, precipitated matter at bottom having characteristic odour

Specific gravity at 25° C.: 1.05-1.07

Total solids: 15-20% w/v

pH at 25° C.: 3.5-4.00

Alcohol content: 10-12% v/v

Reducing sugars: 14-18%

Non-reducing sugars: <0.5%

Methanol: Absent

Microbial limits: Total Viable Count (TVC)—NMT 10⁵/gm
: Yeast and Moulds—NMT 10³/gm
: E. coli—Absent

Heavy metal limits: Not more than 20 ppm

Thin Layer Chromatography in Toluene: Ethyl acetate: Formic acid: 6:3:1
: UV 254 nm—06 spots Rf: 0.20 (Violet), 0.28, 0.40, 0.49, 0.56, 0.74 (All blue)
: UV 366 nm—04 spots Rf: 0.025 (Blue), 0.20, 0.28, 0.40 (All yellow)
: 1% Ethanolic potassium hydroxide reagent—03 spots Rf: 0.20 (Brown), 0.40 (Grey), 0.74 (Brown)

### Example 2

### Efficacy Study of the Herbo-Mineral Metallic Pharmaceutical Kit of the Present Disclosure:

In this study, 9 breast cancer patients carrying BRCA1/2 gene mutation were included. These patients were treated with herbo-mineral metallic pharmaceutical kit after completion of their conventional treatment.

Inclusion Criteria for Enrolling Patients in this Study

Female patients: breast cancer patients of all stages in age group between 25-70 years carrying BRCA1/2 gene mutation

Exclusion Criteria for Enrolling Patients in this Study

Patients who were on other Ayurvedic drugs for breast cancer or any other ailment and patients who had distant metastasis and recurrence.

Demographic Data of Patients Enrolled in this Study

The demographic details of patients enrolled in this study are provided in Table 8 below.

TABLE 8
Demographic data of patients enrolled in this study

| | | No. of |
|---|---|---|
| | | patients |
| Parameters | | (n = 9) |
| Age group (In yrs.) between - | 25-30 | 1 |
| | 30-40 | 4 |
| | 40-50 | 4 |
| Triple Negative (ER, PR, Her 2 negative) | | 9 |
| BRCA mutation | BRCA 1 mutation | 7 |
| | BRCA 2 mutation | 2 |
| Grade | II | 5 |
| | III | 4 |
| Stage | I | 1 |
| | II | 4 |
| | III | 3 |
| | IV | 1 |
| Family History of Cancer | Positive | 7 |
| | Negative | 2 |
| Breast Conservative surgery of affected | Done | 5 |
| breast | | |
| | Not Done | 4 |
| Chemotherapy | Taken | 9 |
| | Not Taken | 0 |
| Radiotherapy | Taken | 8 |
| | Not Taken | 1 |
| Prophylactic Contralateral Breast Surgery | Done | 2 |
| | Not Done | 7 |
| Prophylactic Oophorectomy | Done | 4 |
| | Not Done | 5 |

### Outcome Measures—

### Time Points for Assessment of Outcome Measures—

A—Before starting herbo-mineral metallic pharmaceutical kit (n=9)
B—1 year after starting herbo-mineral metallic pharmaceutical kit (n=9)
C—2 years after starting herbo-mineral metallic pharmaceutical kit (n=6)
D—3 years after starting herbo-mineral metallic pharmaceutical kit (n=3)

### A. Clinical Investigations

The Patients were Followed for:
*Assessment of performance status using Karnofsky score (Grading for well-being on 0 to 100 scale, higher score denotes better performance) and Weight.
*Assessment of Quality of Life (QoL) using Questionnaire QLQ C30 (designed for all types of cancers) and BR 23 (specially designed for breast cancer patients) of EORTC determined on the basis of patients' own perspective about her well-being.
QLQ C30 can be interpreted as—
1. Symptomatology (Symptom score)
2. Ability to perform routine activities (Functional score)
3. Overall well-being (Global score)

(Karnofsky score and scoring for QoL are internationally accepted means of scoring symptoms and quality of life for cancer patients used in various studies in clinical trials.)

To support the clinical observations, additional studies were conducted using clinical laboratory investigations and basic laboratory investigations as follows:

### B. Clinical Laboratory Investigations:

These investigations were conducted for assessing hemoglobin status along with toxicity for liver and kidney on the basis of corresponding enzyme levels. CA 125—an ovarian cancer specific tumor marker was studied to assess tumor burden (in view of higher chances of developing ovarian cancers in BRCA 1/2 mutated breast cancer patients). CRP levels were assessed as an indicator of initiation of inflammatory response.

The tests performed were
Hemogram
LFT (Liver Function Test)
KFT (Kidney Function Test)
Tumor marker CA 125
CRP Test

### C. Basic Laboratory Investigations:

These tests were carried out to provide proof of concept by assessing status of immunomodulatory effect and oxidative stress management in these patients. Cytokines are the key molecules controlling proliferation, differentiation, and functions of immune cells and inflammatory response. Immunological investigations—pro inflammatory cytokines—IL-1β, IL-6, IL-8 and IL-10 assessed by ELISA, using commercial kits.

The oxidative stress is considered to be involved in the pathophysiology of cancers. The activity of erythrocyte antioxidant defense enzymes superoxide dismutase (SOD) and catalase, and Glutathione were studied by colorimetric assays, using commercial kits.

The data for biochemical investigations is represented as absolute values at each time point while for basic laboratory parameters and QLQ assessment, data was analyzed as fold change in a given parameter compared to its level at time point A.

For symptoms Mann Whitney Z test and for scores and basic and clinical laboratory investigations paired ‘t’ test were applied.

### Example 3: Efficacy Studies of the Herbo-Mineral Metallic Pharmaceutical Kit Using Hematological and Biochemical Parameters

The clinical laboratory parameters studied were Hemogram, LFT, KFT, CRP, and tumour marker CA 125. All these parameters were within normal range. Given below were the trends at different time points. The results are based on mean values at each time point.

### Hematological Parameters—

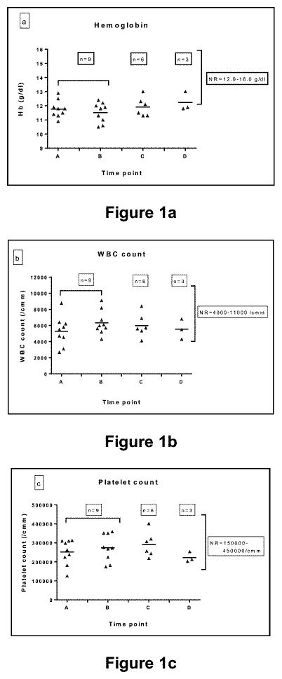

Haemoglobin and WBC—Heamoglobin levels were either within or close to the normal range for all the patients. WBC counts were in normal range for most of the patients throughout the treatment. Only in two cases WBC was below normal range before treatment which improved to the normal range (NR) at the end of one year of treatment (FIG. 1a & b).

Platelets—Platelet counts were in the normal range for all the patients throughout the treatment. Only in two cases it was below normal range (NR) at time point A, which improved to normal range within one year of treatment (FIG. 1c).

### Biochemical Parameters—

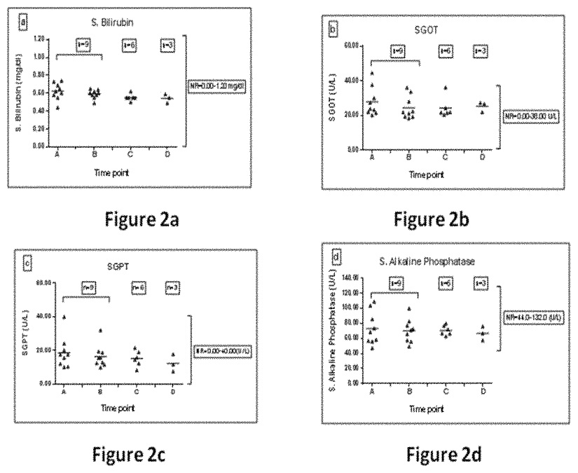

Serum Bilirubin (Total) and S Alkaline Phosphatase—These levels remained within the normal range for all the patients for all four time points (FIGS. 2a and d).

SGOT and SGPT—These levels remained within the normal range for all the patients for all the four time points except for 1 patient. For this patient before treatment both the levels were high, beyond normal range. However, upon treatment for 1 year itself these levels became normal (FIGS. 2b and c)

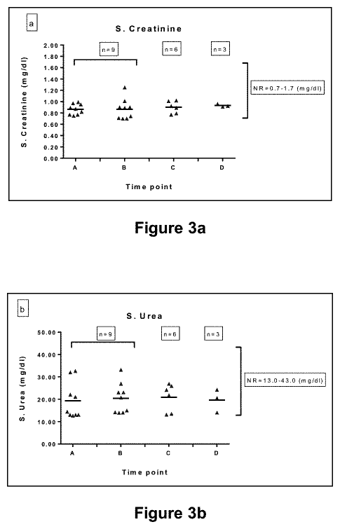

Serum Creatinine and Serum Urea—These levels remained within the normal range for all the patients for all four time points (FIGS. 3a and b).

These studies indicated non toxicity of the treatment to the liver and kidney in the patients.

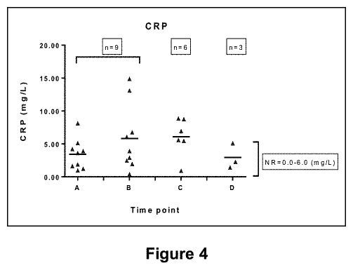

CRP—CRP levels were within the normal range for most of the patients at all the four time points (FIG. 4). However, there were few exceptions as follows—

Two patients revealed higher CRP values (14.88 and 13.01) at time point B i.e. one year after OAM treatment. They were suffering from other symptoms like abdominal pain, fever, cough and cold in one case while knee joint pain along with lower limb pain in the other case at that time point. CRP levels in these patients reduced to normal range (6.9 and 5.6 respectively) at the end of 2 years of treatment.

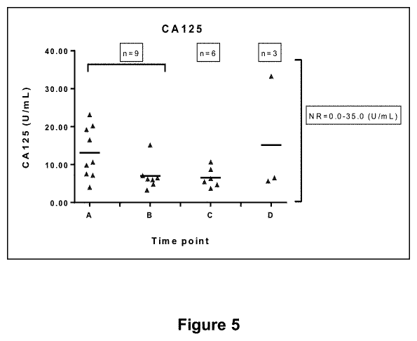

CA125 level—Level of CA 125 is within normal level throughout the treatment. (FIG. 5).

### Example 4: Studies on Immunomodulatory Markers and Oxidative Stress Markers Using Ayurvedic Treatment

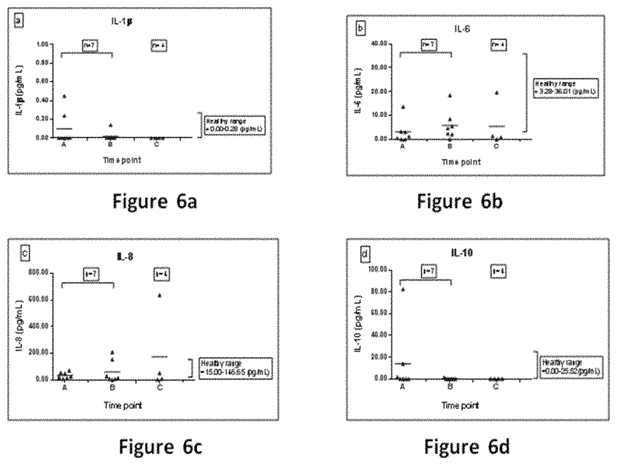

Immunomodulatory markers: As stated before, levels of pro-inflammatory cytokines IL-1β, IL-6, IL-8, and IL-10 were assessed in 7 out of 9 patients to study the pro-inflammatory response (FIGS. 6a, b, c and d). In case of IL-11, and IL-6 all the patients depicted healthy range while in case of IL-8 and IL-10 only one patient each indicated higher levels at time point C and A respectively. Higher IL-8 level at time point C can be correlated with clinical presentation of hyperacidity. There was no disease progression at this time point in radiological assessment. On the other hand 1 patient showed higher level of IL-10 at time point A probably due to generalized weakness and pain in lower extremities just after completion of conventional radiotherapy treatment. However, this patient showed improvement in IL-10 level at 1 year of treatment with herbo-mineral metallic pharmaceutical kit.

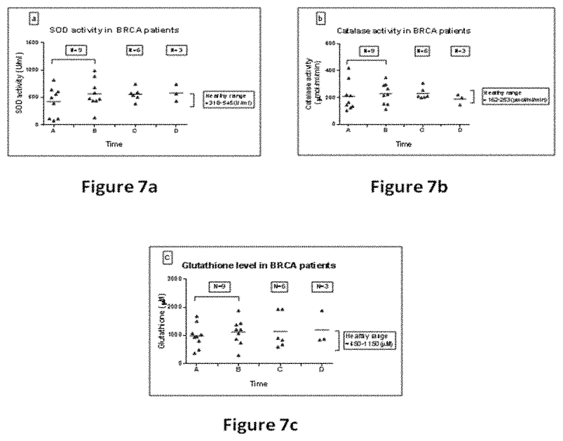

Oxidative Stress markers: Enzymic (Superoxide dismutase and Catalase) and non-enzymic (Glutathione) antioxidant levels in erythrocyte lysates of BRCA positive breast cancer patients were studied to evaluate the antioxidant effect of herbo-mineral metallic pharmaceutical kit. The mean SOD activity and Glutathione levels in these patients increased slightly after 1 year of treatment and remained unchanged thereafter (FIG. 7 a and c). However, Catalase activity in these patients increased after 1 year remained unchanged till second year and decreased slightly thereafter (FIG. 7 b).

### Example 5: Assessment of Quality of Life Using Herbo-Mineral Metallic Pharmaceutical Kit

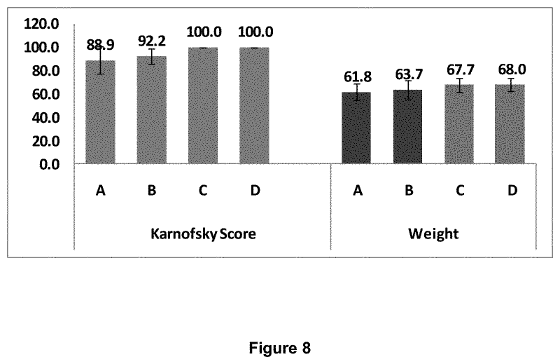

Karnofsky score and Weight—The absolute values of Karnofsky score and Weight have been represented in the form of graphs of mean values at each time point (FIG. 8). Both showed increasing trend at time point B and C indicating improved performance status of the patients upon treatment with herbo-mineral metallic pharmaceutical kit for the period of 1, 2 and 3 years, receptively. Overall improved scores at time points C and D, indicate long-term effectiveness of treatment with the present herbo-mineral metallic pharmaceutical kit (Table 9, FIG. 8).

TABLE 9
Change in Karnofsky score at time-points B, C and
D in BRCA positive breast cancer patients treated
with theherbo-mineral metallic pharmaceutical kit.

| | At time | At time | At time |
|---|---|---|---|
| Change in Karnofsky score | point B | point C | point D |
| compared to time point A | (n = 9) | (n = 6) | (n = 3) |
| Karnofsky score increased by 30 | 0 | 1 | 0 |
| Karnofsky score increased by 20 | 3 | 2 | 1 |
| Karnofsky score increased by 10 | 0 | 1 | 1 |
| Karnofsky score stable | 3 | 2 | 1 |
| Karnofsky score decreased by 10 | 3 | 0 | 0 |
| Karnofsky score decreased by 20 | 0 | 0 | 0 |

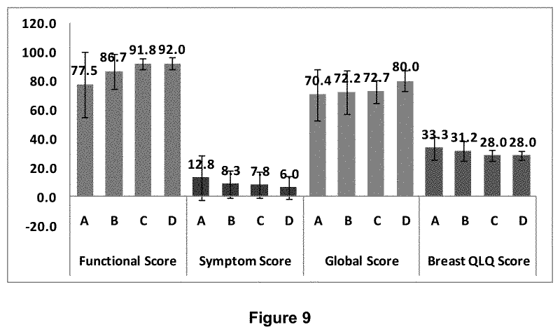

QLQ scores—Functional score also revealed increasing trend while symptom and breast QLQ score showed decreasing trends at time points B, C and D. Moreover, global score remained unchanged. All these trends in four scores indicated improved quality of life in these patients (FIG. 9).

### Example 6: Assessment of Cancer Status with Radiological Investigations (PET CT) in BRCA Mutation Positive Breast Cancer Patients Treated with the Herbo-Mineral Metallic Pharmaceutical Kit

TABLE 10
Radiological assessment of BRCA mutation
positive breast cancer patients

| No. of | Completed | |
|---|---|---|
| Patients | yrs of | |
| (n = 9) | treatment | Treatment response |
| 3 | 3 yrs | Disease-free (No metabolic activity anywhere in |
| | | the body as per PET CT scan till the end of 3 |
| | | yrs). |
| 2 | 2 yrs | Disease-free (No metabolic activity anywhere in |
| | | the body as per PET CT scan till the end of 2 |
| | | yrs). |
| 1 | 1 Yr & | Patient expired due to progressive disease, after |
| | 10 months | 1 year 10 months of starting Ayurvedic treatment |
| 3 | 1 yr | Disease-free (No metabolic activity anywhere in |
| | | the body as per PET CT scan till end of 1 yr). |

A remarkable finding as seen from Table 10 is that 8 out of 9 patients treated with the herbo-mineral metallic pharmaceutical kit were disease free after 1 year, whereas 6 out of 9 patients were disease free after 2 years of Ayurvedic treatment. Remaining 2 are still under Ayurvedic treatment with improved QoL and will complete 2 years of Ayurvedic treatment in near future. 3 patients have completed 3 years of Ayurvedic treatment and are disease free.

As mentioned in demographic data (Table 8), 2 out of 9 and 4 out of 9 patients have undergone prophylactic collateral breast surgery and oophorectomy respectively. However premature menopause and its associated effect on quality of life, bone health and several other parameters are reported in salphingo oophorectomy. Secondly, in case of contralateral prophylactic mastectomy the breast cancer risk is reported to be reduced to 75% to 95%. Even then the herbo-mineral metallic pharmaceutical kit has been helpful in maintaining healthy status of the patients and maintaining disease free survival in all these patients. Remaining patients also showed healthy status even after not undergoing these prophylactic surgeries and good quality of life was maintained with the treatment of herbo-mineral metallic pharmaceutical kit.

No cancer related complaints were reported in any of these patients throughout the study period except in one patient. This patient was initially diagnosed with breast cancer and completed conventional cancer treatment like surgery followed by chemotherapy. After 6 years she suffered with cystadenocarcinoma of ovaries and was enrolled for the current study in stage IV of the disease. She also had metastasis in mediastinal and Rt supraclavicular nodes. This patient survived up to 1 year of the study.

Overall, it is evident that the herbo-mineral metallic pharmaceutical kit is leading to better quality of life. It is further evident, based on Karnofsky score, Functional score, Symptom score, Global score and Breast sore that the quality of life improved in these patients. Also stable heamatological, biochemical and immunological parameters and disease free status in radiological investigations indicate healthy status of the breast cancer patients with BRCA 1/2 mutations.

### TECHNICAL ADVANCEMENTS

The present disclosure described herein above has several technical advantages including, but not limited to, the realization of a herbo-mineral metallic pharmaceutical kit that can:
Delay and/or control recurrence or occurrence of breast and/or ovarian cancer in second breast and/or ovary or metastasis in genetically predisposed breast and ovarian cancer patients;
provide disease-free survival and overall survival to genetically predisposed breast and ovarian cancer patients
maintain biochemical and clinical health of genetically predisposed human individuals having breast and ovarian cancers.
reduce oxidative stress in genetically predisposed breast and ovarian cancer patients
improve the immune response in genetically predisposed breast and ovarian cancer patients
improve the quality of life in genetically predisposed breast and ovarian cancer patients.

The embodiments as described herein above, and various features and advantageous details thereof are explained with reference to the non-limiting embodiments in the description. Descriptions of well-known aspects, components, and molecular biology techniques are omitted so as to not unnecessarily obscure the embodiments herein.

The foregoing description of specific embodiments so fully reveal the general nature of the embodiments herein, that others can, by applying current knowledge, readily modify and/or adapt for various applications of such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein. Further, it is to be distinctly understood that the foregoing descriptive matter is to be interpreted merely as illustrative of the disclosure and not as a limitation.

Having described and illustrated the principles of the present disclosure with reference to the described embodiments, it will be recognized that the described embodiments can be modified in arrangement and detail without departing from the scope of such principles.

While considerable emphasis has been placed herein on the particular features of this disclosure, it will be appreciated that various modifications can be made, and that many changes can be made in the preferred embodiment without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

1. A herbo-mineral metallic pharmaceutical kit, for delaying and/or controlling the recurrence or occurrence of breast and/or ovarian cancer, said kit comprising:
a. a first container containing Suvarna Bhasmadi Vati (SBV) in solid dosage form in an amount in the range of 300 mg/day to 500 mg/day;
b. a second container containing Mouktikyukta Kamdudha Vati (MKV) in solid dosage form in an amount in the range of 800 mg/day to 1200 mg/day;
c. a third container containing Trailokyachintamani Rasa Vati (TCR) in solid dosage form in an amount in the range of 200 mg/day to 300 mg/day;
d. a fourth container containing Kumari Kalpa Vati (KKV) in solid dosage form in an amount in the range of 1600 mg/day to 2400 mg/day; and
e. a fifth container containing Khadirarishta (KHA) in liquid dosage form in an amount in the range of 10 ml/day to 30 ml/day.

2. The kit as claimed in claim 1, wherein said Suvarna Bhasmadi Vati (SBV) is prepared from:
Suvarna bhasma in an amount ranging from 2 wt % to 7 wt % of the total weight of said SBV;
Mouktik bhasma in an amount ranging from 20 wt % to 35 wt % of the total weight of said SBV;
Guduchisattva in an amount ranging from 45 wt % to 60 wt % of the total weight of said SBV; and
at least one first excipient in an amount ranging from 5 wt % to 30 wt % of the total weight of said SBV.

3. The kit as claimed in claim 1, wherein said Mouktikyukta Kamdudha Vati (MKV) is prepared from:
Mouktik bhasma in an amount ranging from 10 wt % to 14 wt % of the total weight of said MKV;
Shankha bhasma in an amount ranging from 10 wt % to 14 wt % of the total weight of said MKV;
Shouktik bhasma in an amount ranging from 10 wt % to 14 wt % of the total weight of said MKV;
Kapardik bhasma in an amount ranging from 10 wt % to 14 wt % of the total weight of said MKV;
Praval bhasma in an amount ranging from 10 wt % to 14 wt % of the total weight of said MKV;
Guduchisattva in an amount ranging from 10 wt % to 14 wt % of the total weight of said MKV;
Shuddha Gairik in an amount ranging from 10 wt % to 14 wt % of the total weight of said MKV; and
at least one second excipient in an amount ranging from 10 wt % to 30 wt % of the total weight of said MKV.

4. The kit as claimed in claim 1, wherein said Trailokyachintamani Rasa Vati (TCR) is prepared from:
a first powder comprising
Kajjali in an amount ranging from 0.012 wt % to 0.016 wt % of the total weight of said TCR;
Suvarna bhasma in an amount ranging from 0.006 wt % to 0.008 wt % of the total weight of said TCR;
Roupya bhasma in an amount ranging from 0.006 wt % to 0.008 wt % of the total weight of said TCR;
Heerak bhasma in an amount ranging from 0.006 wt % to 0.008 wt % of the total weight of said TCR;
Loha bhasma in an amount ranging from 0.006 wt % to 0.008 wt % of the total weight of said TCR;
Abhrak bhasma in an amount ranging from 0.006 wt % to 0.008 wt % of the total weight of said TCR;
Tamra bhasma in an amount ranging from 0.006 wt % to 0.008 wt % of the total weight of said TCR;
Shankha bhasma in an amount ranging from 0.006 wt % to 0.008 wt % of the total weight of said TCR;
Mouktik bhasma in an amount ranging from 0.006 wt % to 0.008 wt % of the total weight of said TCR;
Praval bhasma in an amount ranging from 0.006 wt % to 0.008 wt % of the total weight of said TCR;
Shuddha Hartaal in an amount ranging from 0.006 wt % to 0.008 wt % of the total weight of said TCR;
Shuddha Manahshila in an amount ranging from 0.006 wt % to 0.008 wt % of the total weight of said TCR;
a first liquid comprising
decoction of fresh juice and/or latex of plants selected from the group consisting of Arkadugdha, Nirgundi, Surana, Snuhi Kshir, and Chitrak, each in an amount ranging from 0.13 to 0.17 v/v % of said TCR;
a second powder comprising
Shuddha Kapardik in an amount ranging from 2.7 wt % to 4.7 wt % of the total weight of said TCR;
a third powder comprising
Rasasindoor in an amount ranging from 3 wt % to 5 wt % of the total weight of said TCR;
Vaikrant bhasma in an amount ranging from 0.75 wt % to 1.25 wt % of the total weight of said TCR;
a second liquid comprising
decoction of Chitrak in an amount ranging from 54.5 wt % to 56 wt % of said TCR;
fresh juice of Shigruin an amount ranging from 54.5 wt % to 56 wt % of said TCR;
fresh juice of Ardrak in an amount ranging from 54.5 wt % to 56 wt % of said TCR; and
fresh juice of Nimbuk in an amount ranging from 54.5 wt % to 56 wt % of said TCR;
a fourth powder comprising
Shuddha Tankan in an amount ranging from 5.1 wt % to 7.1 wt % of the total weight of said TCR;
Shuddha Vatsanabh in an amount ranging from 5.1 wt % to 7.1 wt % of the total weight of said TCR;
Marich powder in an amount ranging from 5.1 wt % to 7.1 wt % of the total weight of said TCR;
Suntha powder in an amount ranging from 1.4 wt % to 1.6 wt % of the total weight of said TCR;
Pippali powder in an amount ranging from 1.4 wt % to 1.6 wt % of the total weight of said TCR;
Lavang powder in an amount ranging from 1.4 wt % to 1.6 wt % of the total weight of said TCR;
Jatiphal powder in an amount ranging from 1.4 wt % to 1.6 wt % of the total weight of said TCR;
Haritaki powder in an amount ranging from 1.4 wt % to 1.6 wt % of the total weight of said TCR;
A third liquid comprising
Ardrak fresh juice in an amount ranging from 30.75% to 34.75% v/v of said TCR; and
Nimbuk fresh juice in an amount ranging from 48.75% to 52.75% v/v of said TCR;
at least one third excipient in an amount ranging from 8 wt % to 38 wt % of the total weight of said TCR.

5. The kit as claimed in claim 1, wherein said Kumari Kalpa Vati (KKV) is prepared from:
a powdered mixture comprising
Shunthi powder in an amount ranging from 7 wt % to 9 wt % of the total weight of said KKV;
Marich powder in an amount ranging from 7 wt % to 9 wt % of the total weight of said KKV;
Pippali powder in an amount ranging from 7 wt % to 9 wt % of the total weight of said KKV;
Bhumiamalaki powder in an amount ranging from 7 wt % to 9 wt % of the total weight of said KKV;
Bibhitaki powder in an amount ranging from 7 wt % to 9 wt % of the total weight of said KKV;
Haritaki powder in an amount ranging from 7 wt % to 9 wt % of the total weight of said KKV;
Musta powder in an amount ranging from 7 wt % to 9 wt % of the total weight of said KKV;
Vidang powder in an amount ranging from 7 wt % to 9 wt % of the total weight of said KKV;
Sharpunkha powder in an amount ranging from 7 wt % to 9 wt % of the total weight of said KKV; and
Kutki powder in an amount ranging from 7 wt % to 9 wt % of the total weight of said KKV;
a ground pulp comprising
Kumari in an amount ranging from 1.75 wt % to 2.25 wt % on dry basis of the total weight of said KKV; and
at least one fourth excipient in an amount ranging from 8 wt % to 28 wt % of the total weight of said KKV.

6. The kit as claimed in claim 1, wherein said Khadirarishta (KHA) is prepared from:
A first powder comprising
Khadir coarse powder in an amount ranging from 8.75 wt % to 12.75 wt % of the total weight of said KHA;
Devdaru coarse powder in an amount ranging from 8.75 wt % to 12.75 wt % of the total weight of said KHA;
Daruharidra coarse powder in an amount ranging from 2.3 wt % to 6.3 wt % of the total weight of said KHA;
Haritaki coarse powder in an amount ranging from 1 wt % to 1.5 wt % of the total weight of said KHA;
Bibhitaki coarse powder in an amount ranging from 1 wt % to 1.5 wt % of the total weight of said KHA;
Amalaki coarse powder in an amount ranging from 1 wt % to 1.5 wt % of the total weight of said KHA;
Jaggery in an amount ranging from 60 wt % to 70 wt % of the total weight of said KHA;
Dhataki flowers as a natural inoculum, in an amount ranging from 3.1 wt % to 3.5 wt % of the total weight of said KHA;
a second fine powder comprising
Jatiphal powder in an amount ranging from 0.19 wt % to 0.23 wt % of the total weight of said KHA;
Tamalpatra powder in an amount ranging from 0.19 wt % to 0.23 wt % of the total weight of said KHA;
Bruhad Ela powder in an amount ranging from 0.19 wt % to 0.23 wt % of the total weight of said KHA;
Lavang powder in an amount ranging from 0.19 wt % to 0.23 wt % of the total weight of said KHA;
Dalchini powder in an amount ranging from 0.19 wt % to 0.23 wt % of the total weight of said KHA;
Kankol powder in an amount ranging from 0.19 wt % to 0.23 wt % of the total weight of said KHA;
Nagkeshar powder in an amount ranging from 0.19 wt % to 0.23 wt % of the total weight of said KHA; and
Pippali powder in an amount ranging from 0.19 wt % to 0.23 wt % of the total weight of said KHA.

7. A process of preparing a herbo-mineral metallic pharmaceutical kit, said process comprising the following steps:
A. preparing Suvarna Bhasmadi Vati (SBV) in solid dosage form by
i. mixing Suvarna bhasma, Mouktik bhasma, Guduchi sattva, and at least one first excipient with water to obtain a dough; wherein said Suvarna bhasma is obtained by triturating incinerated Suvarna with fresh juice of the leaves of Ocimum sanctum;
ii. pelletizing said dough to obtain pellets;
iii. drying said pellets at 45° C. to obtain dried pellets;
iv. grinding said dried pellets to obtain a first mixture having powder to granule ratio of 30:70; and
v. compressing said first mixture to obtain Suvarna Bhasmadi Vati (SBV) in solid dosage form.
B. preparing Mouktikyukta Kamdudha Vati (MKV) in solid dosage form by
i. mixing Mouktik bhasma, Shankha bhasma, Shouktik bhasma, Kapardik bhasma, Praval bhasma, Guduchi sattva, Shuddha Gairik and at least one second excipient with water to obtain a dough;
ii. pelletizing said dough to obtain pellets;
iii. drying said pellets at 45° C. to obtain dried pellets;
iv. grinding said dried pellets to obtain a second mixture having powder to granule ratio of 30:70; and
v. compressing said second mixture to obtain Mouktikyukta Kamdudha Vati (MKV) in solid dosage form.
C. preparing Trailokyachintamani Rasa Vati (TCR) in solid dosage form by
i. mixing and powdering Kajjali, Suvarna bhasma, Roupya bhasma, Heerak bhasma, Loha bhasma, Abhrak bhasma, Tamra bhasma, Shankha bhasma, Mouktik bhasma, Praval bhasma, Shuddha Hartaal and Shuddha Manahshila, to obtain a first powder;
ii. triturating said first powder sequentially with a first liquid comprising decoction of fresh juice and/or latex of plants selected from the group consisting of Arkadugdha, Nirgundi, Surana, Snuhi Kshir, and Chitrak, to obtain a mixture;
iii. drying said mixture at 45° C. and powdering said dried mixture to obtain a first powdered product;
iv. mixing said first powdered product with a second powder comprising Shuddha Kapardik, and drying overnight at room temperature to obtain a dried material;
v. placing said dried material in earthen vessels and sealing using clay to obtain a sealed arrangement;
vi. incinerating said sealed arrangement at 700-750° C., allowing the incinerated material to cool, and powdering said cooled incinerated material to obtain a second powdered product;
vii. mixing said second powdered product with a third powder comprising Rasasindoor and Vaikrant bhasma and grinding for a period in the range of 2-4 hrs. to obtain a third mixture;
viii. triturating said third mixture sequentially with a second liquid comprising decoction of Chitrak, fresh juice of Shigruin, fresh juice of Ardrak and fresh juice of Nimbuk, to obtain a fourth mixture;
ix. drying said fourth mixture at 45° C. and powdering said dried fourth mixture to obtain a third powdered product;
x. mixing said third powdered product with a fourth powder comprising Shuddha Tankan, Shuddha Bachnag, Marich, Sunthi, Pippali, Lavang, Jayphal, and Haritaki, to obtain a fifth mixture;
xi. triturating said fifth mixture sequentially with a third liquid comprising Nimbuk and Ardrak, to obtain a sixth mixture;
xii. drying said sixth mixture at 45° C. and powdering said dried sixth mixture to obtain a fourth powdered product;
xiii. mixing said fourth powdered product with at least one third excipient and water to form a dough and pelletizing said dough to obtain pellets;
xiv. drying said pellets at 45° C. to obtain dried pellets;
xv. grinding said dried pellets to obtain a sixth mixture having powder to granule ratio of 70:30; and
xvi. compressing said sixth mixture to obtain Trailokyachintamani Rasa Vati (TCR) in solid dosage form.
D. preparing Kumari Kalpa Vati (KKV) in solid dosage form by
i. mixing a powdered mixture comprising Shunthi powder, Marich powder, Pippali powder, Bhumiamalaki powder, Bibhitaki powder, Haritaki powder, Musta powder, Vidang powder, Sharpunkha powder, Kutki powder, and at least one fourth excipient in a ratio of 5:1 to obtain a first mixture;
ii. mixing said first mixture with a ground pulp comprising Kumari, in the ratio of 1:2 (w/w), to obtain a dough;
iii. pelletizing said dough followed by drying at 45° C. to obtain dried pellets;
iv. grinding said dried pellets to obtain a second mixture having powder to granule ratio of 70:30
v. compressing said second mixture to obtain Kumari Kalpa Vati (KKV) in solid dosage form.
E. preparing Khadirarishta (KHA) in liquid dosage form by
i. soaking overnight in water a first powder comprising Khadir coarse powder, Devdaru coarse powder, Daruharidra coarse powder, Haritaki coarse powder, Bibhitaki coarse powder, Amalaki coarse powder, and heating to obtain a decoction;
ii. filtering said decoction to obtain a filtrate and allowing said filtrate to cool;
iii. adding crushed jaggery to said filtrate and dissolving completely to obtain a slurry;
iv. filtering said slurry and mixing with Dhataki flowers as natural inoculum and with a second fine powder comprising Jatiphal powder, Tamalpatra powder, Bruhad Ela powder, Lavang powder, Dalchini powder, Kankol powder, Nagkeshar powder, Pippali powder, to obtain a first mixture;
iv. incubating said first mixture in a closed barrel at a temperature in the range of 32-34° C. for a period of 21 to 30 days to obtain a fermented mixture;
v. filtering the fermented mixture to obtain Khadirarishta (KHA) in liquid dosage form

8. The kit as claimed in claim 1, wherein said Suvarna Bhasmadi Vati (SBV), said Mouktikyukta Kamdudha Vati (MKV), said Trailokyachintamani Rasa Vati (TCR) and said Kumari Kalpa Vati (KKV) are in solid dosage form selected from the group consisting of tablet, pill and capsule.

9. The kit as claimed in claim 1, wherein said Khadirarishta (KHA) is in liquid dosage form, comprising at least one liquid selected from the group consisting of water, hydro-alcohol, and alcohol.

10. The kit as claimed in claim 2, wherein said first excipient, said second excipient, said third excipient, and said fourth excipient are independently selected from the group consisting of gum acacia, guar gum and xanthan gum.

11. The kit as claimed in claim 3, wherein said first excipient, said second excipient, said third excipient, and said fourth excipient are independently selected from the group consisting of gum acacia, guar gum and xanthan gum.

12. The kit as claimed in claim 4, wherein said first excipient, said second excipient, said third excipient, and said fourth excipient are independently selected from the group consisting of gum acacia, guar gum and xanthan gum.

13. The kit as claimed in claim 5, wherein said first excipient, said second excipient, said third excipient, and said fourth excipient are independently selected from the group consisting of gum acacia, guar gum and xanthan gum.
